(19) United States
(12) Patent Application Publication
(10) Pub. No.: US 2018/0263488 A1
(43) Pub. Date: Sep. 20, 2018

(54) Variable Lens System for Refractive Measurement

(71) Applicant: EYENETRA, INC., Somerville, MA (US)

(72) Inventors: Vitor PAMPLONA, Somerville, MA (US); Nathaniel SHARPE, Cambridge, MA (US); Matthias HOFMANN, Wayland, MA (US); Monica Mitiko Soares MATSUMOTO, Somerville, MA (US)

(21) Appl. No.: 15/541,453

(22) PCT Filed: Jan. 13, 2016
(86) PCT No.: PCT/US2016/013304

§ 371 Date: Jul. 3, 2017

Related U.S. Application Data
(63) Continuation-in-part of application No. PCT/US2015/022138, filed on Mar. 24, 2015.
(60) Provisional application No. 62/103,062, filed on Jan. 13, 2015.

(51) Int. Cl. A61B 3/028; A61B 3/00

(52) U.S. Cl. CPC A61B 3/028; A61B 3/0041; A61B 3/0008; A61B 3/0025

(57) ABSTRACT

A refractive measurement tool measures refractive aberrations of the human eye. The tool includes a variable lens system. One or more refractive attributes of the variable lens system—such as spherical power, cylindrical power, cylindrical axis, prism or base—are adjustable. The user holds the tool up to his or her eyes, and looks through the tool at a near or far scene. Iterative vision tests are performed, in which refractive properties of the variable lens system are changed from iteration to iteration.

This application claims the priority of International Application Number PCT/US2015/022138, international filing date Mar. 24, 2015, titled Methods and Apparatus for Optical Controller, the entire disclosure of which is herein incorporated by reference.

This application is a non-provisional of, and claims the priority of, U.S. Provisional Patent Application No. 62/103,062, filed Jan. 13, 2015, the entire disclosure of which is herein incorporated by reference.

### FIELD OF TECHNOLOGY

The present invention relates generally to assessment of refractive aberrations.

### SUMMARY

In illustrative implementations of this invention, a refractive measurement tool measures refractive aberrations of the human eye. The tool includes a variable lens system (VLS). One or more refractive attributes (e.g., spherical power, cylindrical power, cylindrical axis, prism or base) of the VLS are adjustable. The user holds the tool up to his or her eyes, and looks through the tool (including through the VLS) at a near or far scene. Iterative vision tests are performed, in which refractive properties of the VLS are changed from iteration to iteration. I/O devices receive input from the user regarding which VLS setting results in clearer vision. For example, in some use scenarios, if spherical power is being optimized during a particular step of the testing procedure, the user inputs feedback regarding whether a test image appears clearer with the current VLS setting (a changed spherical power) than with the last VLS setting (a prior spherical power).

In illustrative implementations, one or more computers calculate a refractive assessment of the eye being tested. The refractive assessment includes one or more of the following, for each of the user's eyes: (a) a spherical correction to correct a spherical aberration of the eye; (b) a cylindrical correction (e.g., axial parameter correction) to correct a cylindrical aberration of the eye; and (c) a prismatic correction to correct a prismatic aberration of the eye. The refractive assessment also includes pupillary distance (PD), which is also known as inter-pupillary distance. In some cases, the refractive assessment also includes back vertex distance. In illustrative implementations, for a refractive assessment: (a) the spherical correction, cylindrical correction and pupillary distance are calculated separately for near vision and distance vision; (b) the power of spherical and cylindrical corrections are expressed in diopters; (c) the prism correction is expressed in diopters; and (d) either a plus-cylinder or a minus-cylinder notation is used. As can be seen, the content of the refractive assessment may be similar to that of a prescription for eyeglasses or contact lenses. In some cases, the cylindrical correction is expressed as a cylindrical power and a cylindrical axis. In other cases, the cylindrical correction is expressed as a linear combination of two cross-cylindrical lenses (e.g., Jackson cylinders). In some cases, the prismatic correction is expressed in terms of prism (i.e., prismatic displacement of image) and base (direction of the prismatic displacement).

In illustrative implementations, during vision testing, a user attaches a mobile computing device (MCD) to the front of the tool. (The back of the tool is the side of the tool that is held closest to the eyes of the user; the front of the tool is on an opposite side of the tool). In that case, the scene a user sees, when looking through the tool, is an image displayed on a screen of the MCD.

For example, in some cases, the MCD comprises a smartphone or cell phone, and the user views all or portions of the phone's display screen when looking through the tool.

After the MCD is attached to the front of the refractive measurement tool, the user looks through the tool. Specifically, the user holds a viewport (e.g., window) of the tool at eye level, and looks through the tool to see the MCD screen. The user sees light that travels through the tool: light travels from the MCD screen, then through the tool's variable lens system (VLS), then through the tool's viewport, and then to the eyes. The MCD is attached on one side of the tool; the viewport is on an opposite side of the tool. During at least part of the vision test, the MCD screen displays one or more visual patterns that are used in the test.

In illustrative implementations, a user provides feedback regarding which setting of the variable lens system (VLS) produces the clearest vision for the user. For example, in some use scenarios: (a) in a first trial, a VLS refractive attribute (e.g., spherical power, cylindrical power, cylindrical axis, prism or base) is set to a first value while the user looks through the tool at a test image displayed on the MCD screen; (b) in a second trial, the VLS refractive attribute is set to a second value while the user looks through the tool at the same test image on the MCD screen; and (c) an I/O device accepts input from the user regarding whether the image in the second trial looks clearer or less clear than in the first trial. The format of the input may vary. For example, in some cases, a user simply indicates which image he or she prefers, and this input regarding preference is a proxy for which image appears clearer to the user.

In some implementations of this invention: (a) the refractive measurement tool houses a VLS and a NETRA aberrometer, as defined herein; and (b) refractive aberration of an eye is assessed by taking VLS subjective measurements and NETRA Measurements, as defined herein.

“NETRA Patent” means U.S. Pat. No. 8,783,871 B2, issued Jul. 22, 2014, Near Eye Tool for Refractive Assessment, Vitor Pamplona et al. The entire disclosure of the NETRA Patent is hereby incorporated by reference herein.

“NETRA Measurement” and “Near Eye Measurement” each mean a subjective measurement of refractive aberration of an eye, which measurement is taken by a method that (a) is disclosed in the NETRA Patent and (b) involves altering a display such that a user perceives that two or more images become aligned.

“NETRA aberrometer” and “Near Eye Aberrometer” each mean apparatus configured for taking NETRA Measurements.

In some implementations of this invention: (a) the refractive measurement tool houses a VLS and an EPM aberrometer, as defined herein; and (b) refractive aberration of an eye is assessed by taking VLS subjective measurements and EyeNetra Photorefraction Measurements (EPMs), as defined herein.

“EPM Patent Applications” mean: (a) PCT international patent application PCT/US2014/033693, international filing date Apr. 10, 2014, publication number WO/2014/169148, Methods and Apparatus for Refractive Condition Assessment, Vitor Pamplona et al. (the “693 Application”); and (b) U.S. patent application Ser. No. 14/783,790, Methods and Apparatus for Assessment of Refractive Condition, Vitor Pamplona et al., filed Oct. 9, 2015 (the “790 Application”). The entire disclosure of the 790 Application is hereby incorporated by reference herein. The entire disclosure of the U.S. patent application publication of the 790 Application is hereby incorporated by reference herein. The entire disclosure of the 693 Application is hereby incorporated by reference herein.

“EyeNetra Photorefraction Measurement” and “EPM” each mean an objective measurement of refractive aberration of an eye, which objective measurement is taken by a method that is described in the EPM Patent Applications and that involves an out-of-focus image being captured by an imaging system, which imaging system is external to the eye.

“EPM aberrometer” and “EyeNetra Photorefraction Aberrometer” each mean an aberrometer disclosed in the EPM Patent Application.

The description of the present invention in the Summary and Abstract sections hereof is just a summary. It is intended only to give a general introduction to some illustrative implementations of this invention. It does not describe all of the details and variations of this invention. Likewise, the description of this invention in the Field of Technology section is not limiting; instead it identifies, in a general, non-exclusive manner, a technology to which exemplary implementations of this invention generally relate. Likewise, the Title of this document does not limit the invention in any way; instead the Title is merely a general, non-exclusive way of referring to this invention. This invention may be implemented in many other ways.

### BRIEF DESCRIPTION OF THE DRAWINGS

FIG. 1 shows a refractive measurement tool that includes a variable lens system.

FIGS. 2A-2G show examples of variable lenses. FIGS. 2A, 2B and 2C show Alvarez lenses in three different positions. FIG. 2D shows a spherocylindrical Jackson cross-cylinder lens. FIG. 2E shows a Humphrey lens configuration that includes three lens pairs. FIG. 2F show a Barbero-Rubenstein lens configuration that includes two lens pairs. FIG. 2G shows a liquid lens.

FIG. 2H shows a pair of Risley prisms for deflecting a light beam, in order to compensate for differences in inter-pupillary distance (PD).

FIG. 3A shows I/O devices of a refractive measurement tool.

FIG. 3B is a block diagram that shows hardware components of a refractive measurement tool.

FIG. 4 shows an actuator that actuates motion of lenses in a variable lens system relative to each other.

FIGS. 5A and 5B show an actuator for adjusting PD. In FIG. 5A, the actuator is increasing PD. In FIG. 5B, the actuator is reducing PD.

FIG. 6 shows a rack and pinion actuator for adjusting PD.

FIG. 7 shows a swivel joint for adjusting PD.

FIG. 8 shows a monocular device.

FIG. 9A shows a user holding a refractive measurement tool up to his eyes.

FIG. 9B shows a user holding a refractive measurement tool, without an MCD attached.

FIGS. 10A and 10B are each an exploded view of a refractive measurement tool. In FIG. 10A, the tool has a single viewport for both of the user's eyes. In FIG. 10B, the tool has two holes, one hole for each of the user's eyes.

FIGS. 11A, 11B, 11C and 11D are four views of a face-fitting portion of a refractive measurement tool. The face-fitting portion is configured to be pressed against at least the forehead and cheeks of a human user. FIG. 11A is a perspective view; FIG. 11B is a top view; FIG. 11C is a back view; and FIG. 11D is a side view.

FIGS. 12A, 12B, 12C and 12D are four views of an attachment mechanism for attaching a refractive measurement tool to an MCD. FIG. 12A is a perspective view; FIG. 12B is a bottom view; FIG. 12C is a back view; and FIG. 12D is a side view.

FIG. 13A shows a refractive measurement tool attached to straps worn on a head.

FIG. 13B shows a refractive measurement tool attached to headwear worn on a head.

FIG. 13C shows a refractive measurement tool attached to an eyeglass frame.

FIG. 13D shows images displayed on a screen.

FIG. 14 is a block diagram of a refractive measurement tool attached to an MCD.

FIG. 15A shows a refractive assessment tool that includes a variable lens system and an EPM aberrometer.

FIGS. 15B and 15C show steps in a method that includes taking EPMs and taking VLS subjective measurements

FIG. 15D shows steps in another method that includes taking EPMs and taking VLS subjective measurements

FIG. 16A shows a refractive assessment tool that includes a variable lens system and a NETRA aberrometer.

FIG. 16B shows lines in masks in a NETRA aberrometer.

FIG. 17 shows steps in a method that includes taking NETRA Measurements and taking VLS subjective measurements.

FIG. 18 is a flowchart that shows an example of a method for determining the best spherocylindrical refractive correction.

FIGS. 19-26 show steps in methods for refractive measurement.

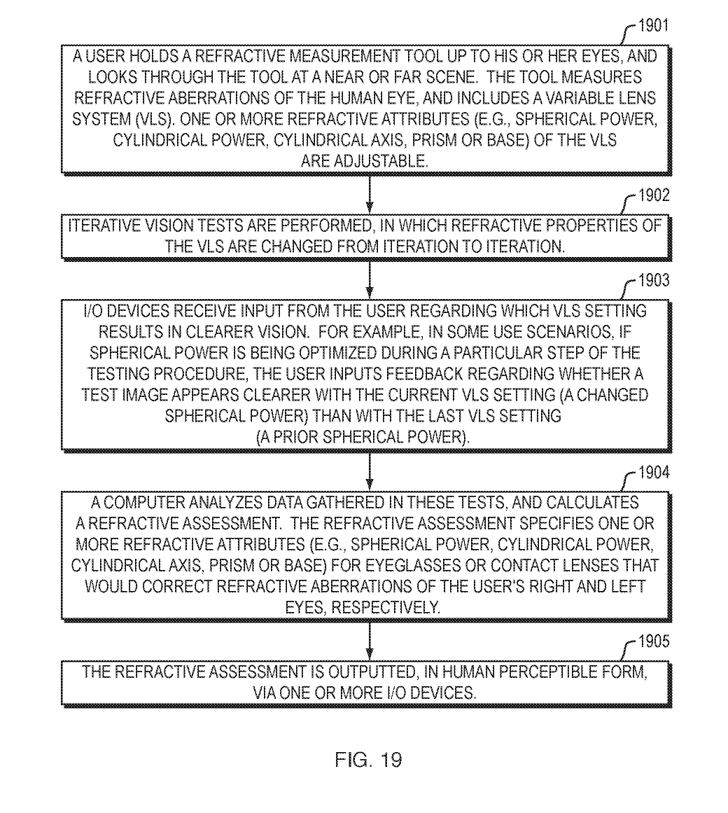

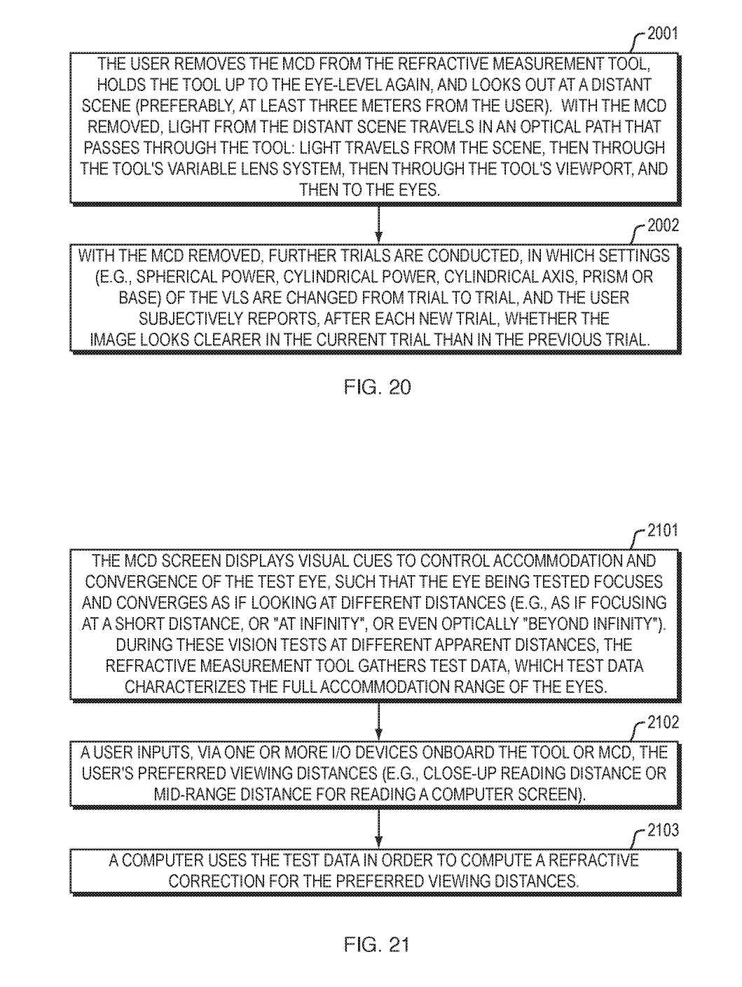

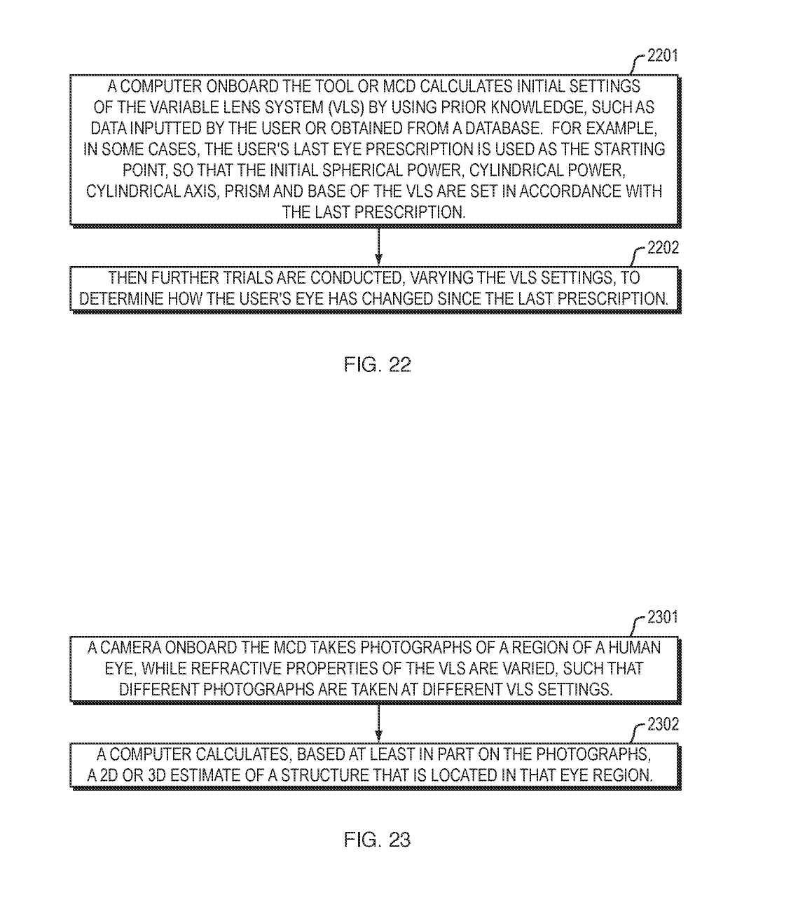

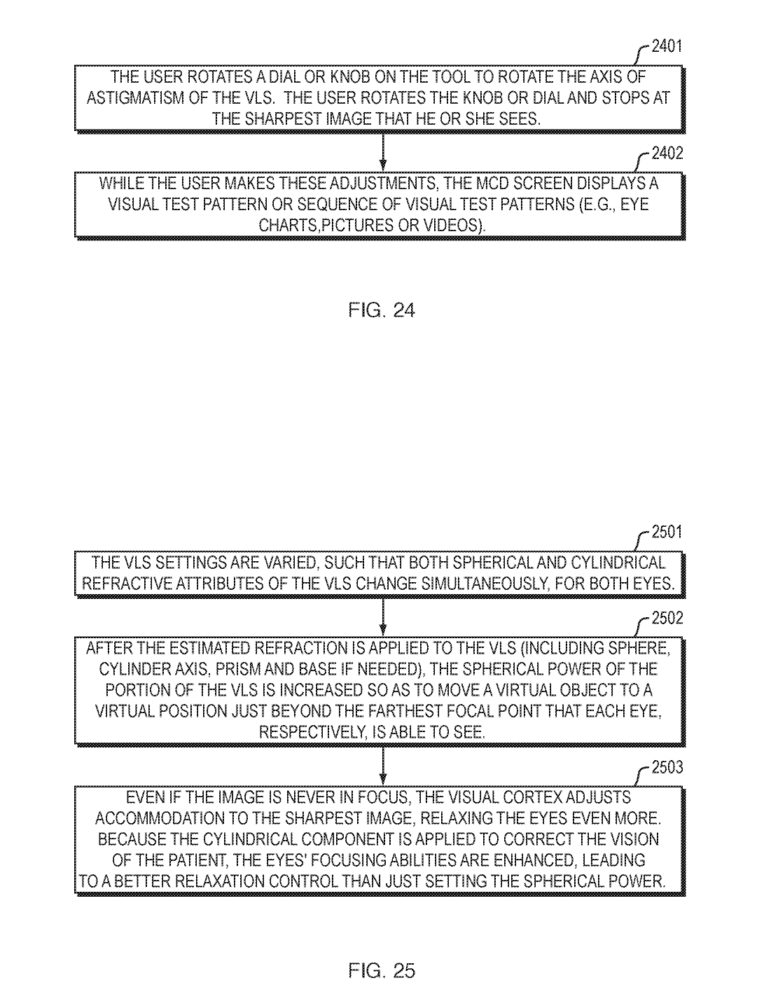

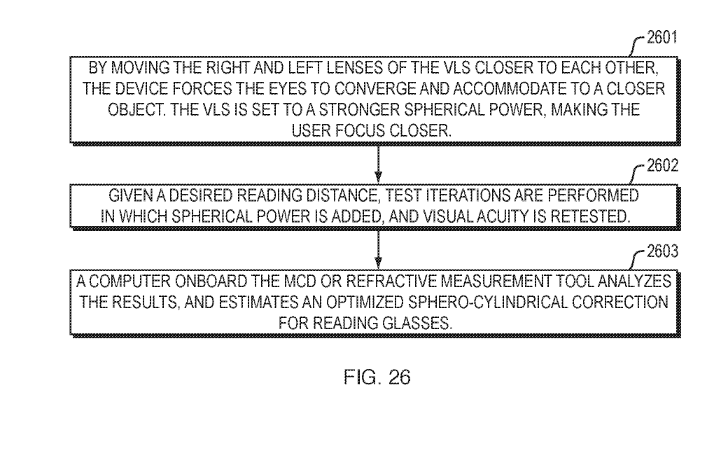

In FIG. 19, the method includes iterative tests with user input. In FIG. 20, the method includes a user removing an MCD from the refractive measurement tool. In FIG. 21, the method includes displaying cues to control accommodation. In FIG. 22, the method includes calculating initial settings of a VLS based on prior knowledge. In FIG. 23, the method includes taking photographs of different regions of an eye at different VLS settings. In FIG. 24, the method includes rotating an axis of astigmatism. In FIG. 25, the method includes adjusting both spherical and cylindrical refractive attributes. In FIG. 26, the method includes controlling convergence.

The above Figures show some illustrative implementations of this invention, or provide information that relates to those implementations. However, this invention may be implemented in many other ways.

### DETAILED DESCRIPTION

### Hardware

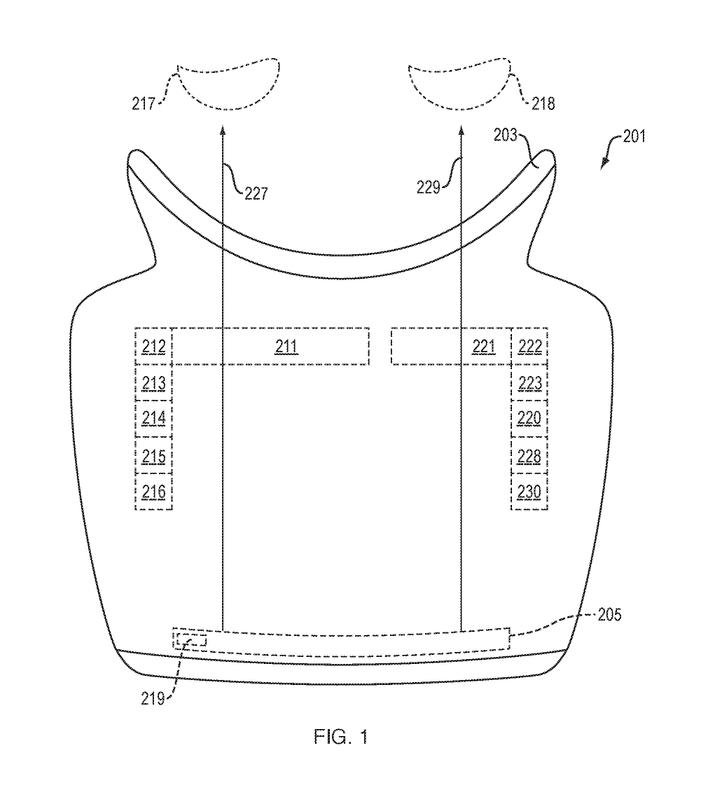

FIG. 1 shows a refractive measurement tool that includes a variable lens system, in an illustrative implementation of this invention. In the example shown in FIG. 1, the refractive measurement tool 201 comprises a handheld bi-ocular device. A mobile computing device (MCD) 205 is attached to the front of the tool, in order to perform a vision check. The user holds the tool up to his or her eyes, and views the MCD display and other elements of the MCD through a view port 203 (e.g., window or eyeshield) in the tool. A computer (e.g., 214) outputs control signals to set the refractive power of the tool's variable lens system and to control an I/O device such that it asks the user to perform a visual test. Feedback from the user is captured either by I/O devices onboard the refractive measurement tool or by the MCD's regular input controls if existing. A user performs a series of tasks, validates and improves his vision on each of the iterations.

In FIG. 1, the refractive measurement tool 201 includes a variable lens system that comprises a right variable lens 211 and a left variable lens 221. The right variable lens 211 refracts light (e.g., 227) bound for a user's right eye 217; the left variable lens 221 refracts light (e.g., 229) bound for a user's left eye 218.

In FIG. 1, the refractive measurement tool 201 includes two electronics modules (EMs) 213, 223 for controlling (a) refractive attributes (e.g., spherical power, cylindrical power, cylindrical axis, prism and base) of the variable lens system (VLS) and (b) the inter-pupillary distance (PD) of bi-ocular lenses of the VLS. The right and left EMs control a right set of one or more actuators 212 and a left set of one or more actuators 222, respectively. In some cases, actuators 212, 222 actuate movement of lenses in the VLS system, in order to adjust one or more refractive attributes of the right and left variable lenses 211, 221. Alternatively or in addition, the actuators 212, 222 actuate motion of binocular parts of the tool, in order to adjust PD.

Alternatively or in addition, the actuators that translate the lenses are in some cases powered by mechanical motion imparted by a human user. For example, in some cases, a user rotates a dial or knob, thereby inputting a mechanical motion that is used to translate lenses or otherwise adjust refractive attributes of the VLS.

A computer 214 in the tool 201 controls the EMs. The computer 214 stores data in, and retrieves data from, a memory device 215. The computer 214 interfaces with a wireless communication module 216 for wireless communication with remote computers. The wireless module 216 includes a wireless receiver, transmitter or transceiver and an antenna. Alternatively, a computer 219 onboard the MCD 205 controls the EMs and one or more actuators in the tool 201. Also, computer 219 onboard the MCD 205 controls a display screen onboard the MCD 205.

The refractive measurement tool includes a power source 220. In some cases, the power source 220 comprises one or more batteries. In other cases, the power source 220 is a wired connection to the MCD. In some alternative cases, the power source 220 includes an adapter, including a rectifier, for rectifying and stepping down voltage from an AC power source, such as a wall outlet.

In illustrative implementations, the mobile computing device (MCD) 205 is an electronic consumer device, such as a smartphone, cell phone, mobile phone, phonepad, tablet, laptop, notebook, notepad, personal digital assistant, enterprise digital assistant, ultra-mobile PC, or other handheld computing device.

In other implementations, the MCD 205 is an electronic device, including a display screen and a computer for controlling the display screen. For example, in some cases, the MCD is custom-built for use with, and is a detachable component of, the refractive measurement tool.

In illustrative implementations, the refractive measurement tool is a lightweight, handheld, bi-ocular device that a user holds up to his or her eyes while conducting a vision testing procedure. In some cases, the MCD 205 is an integral part of the handheld bi-ocular device. In some other cases, the MCD 205 is a separate module. For example, in some cases, the MCD 205 is a separate module that is attached to, or adjacent to, the bi-ocular device.

In illustrative implementations, the tool also calculates back vertex distance (BVD) for corrective eyeglasses—that is, a distance between the eye and an eyeglass lens. Accurate BVD is desirable if a high diopter power is needed for refractive correction. In some cases, the BVD is a fixed constant. In some alternative cases, the tool is adjustable such that the BVD is varied and an appropriate BVD is selected. In these alternative cases, the BVD is adjusted by mechanical motion imparted by the user or by actuators that are controlled by one or more computers (either with or without input from a user). One or more sensors gather sensor data from which the BVD is calculated.

In the example shown in FIG. 1, the variable lens system (VLS) may be implemented in many different ways. For example, in some implementations, the VLS includes one or more of the following: an Alvarez lens pair, Jackson cross-cylinders, Humphrey lenses, a spherocylindrical lens pair, Risley prisms, or liquid lenses. Thus:

In some cases, the VLS includes an Alvarez lens pair, including two lens and one or more actuators. The actuators translate the two Alvarez lenses relative to each other. This translation occurs in one or two dimensions perpendicular to the optical axes of the Alvarez lenses and causes the spherical power, cylindrical power or axis of astigmatism (or a combination of any of these refractive attributes) of the VLS to change. In some cases, an additional actuator rotates the two Alvarez lenses together or relative to each other to facilitate the search for the axis of astigmatism.

In some cases, the VLS includes Jackson cylinders, including two cylindrical lens and an actuator. The actuator rotates one of the cylindrical lens with respect to the other cylindrical lens, thereby varying the cylindrical refraction of the VLS.

In some cases, the VLS includes Humphrey lenses. For example, in some cases, the VLS includes three pairs of lenses (six lenses) and actuators for translating them. The first lens pair is used to adjust spherical power, the second lens pair is used to adjust one cross-cylinder, and the third lens pair is used to adjust the other cross-cylinder. Translation of the six Humphrey lenses thus varies the spherical and cylindrical refraction of the VLS.

In some cases, the VLS includes a spherocylindrical lens pair, including two lenses and actuators for translating them with respect to each other. The translation of the two lenses causes the spherical power and cylindrical refraction of the VLS to change. For example, in some cases, a Barbero-Rubinstein lens pair is used. In that case: (a) the front and rear lenses each have a planar surface and a non-planar surface, with the non-planar surfaces of the front and rear lenses being in contact with each other; (b) the non-planar surface of the front lens is described by

$$u ( x , y ) = A ( \frac{{x}^{3}}{3} + {xy}^{2} ) ,$$

where A is a constant; (c) the non-planar surface of the rear lens is described by

$$v ( x , y ) = B ( \frac{{x}^{3}}{3} - {xy}^{2} ) ,$$

where B is a constant, (d) an actuator translates the front lens along the x direction, and (e) an actuator translates the rear lens along both the x and y directions.

In some cases, the VLS includes two Risley prisms and an actuator for rotating one of the prisms with respect to the other. This rotation changes beam deflection, and thus changes prismatic effects (e.g., prism and base) of the VLS.

In some cases, the VLS includes a liquid lens, the shape of which is controllable via the electrowetting effect by varying an applied voltage or via mechanical pressure on the lens liquids. For example, in some cases, the liquid lens comprises two immiscible liquids, each with a different refractive index, where one of the liquids is an electrically conductive and the other is non-conducting. For example, in some cases, the conducting liquid is an aqueous solution and the non-conducting liquid is an oil.

In some cases, the VLS includes a liquid lens that changes its refractive index radially when voltage is applied, increasing and decreasing the optical power by changing optical properties of the liquid instead of the curvature of the lens.

In some cases, the VLS includes a mechanical system that increases or decreases the amount of liquid inside a lens-shaped transparent chamber. In turn, the increase or decrease of liquid changes the lens chamber's shape, increasing and decreasing optical power.

A wide variety of actuators may be used to translate the lenses. For example, in some cases, the VLS includes one or more linear, rotary, electrical, piezoelectric, or electro-mechanical actuators. These actuators translate one or more lenses in the VLS. In some cases, an actuator includes and is powered by an electrical motor, including any stepper motor or servomotor.

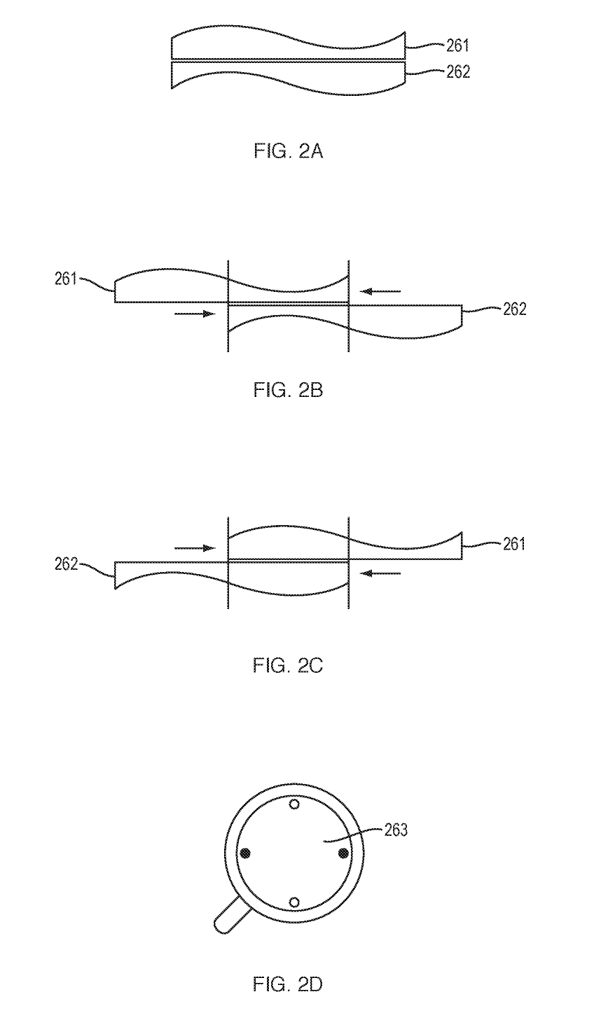

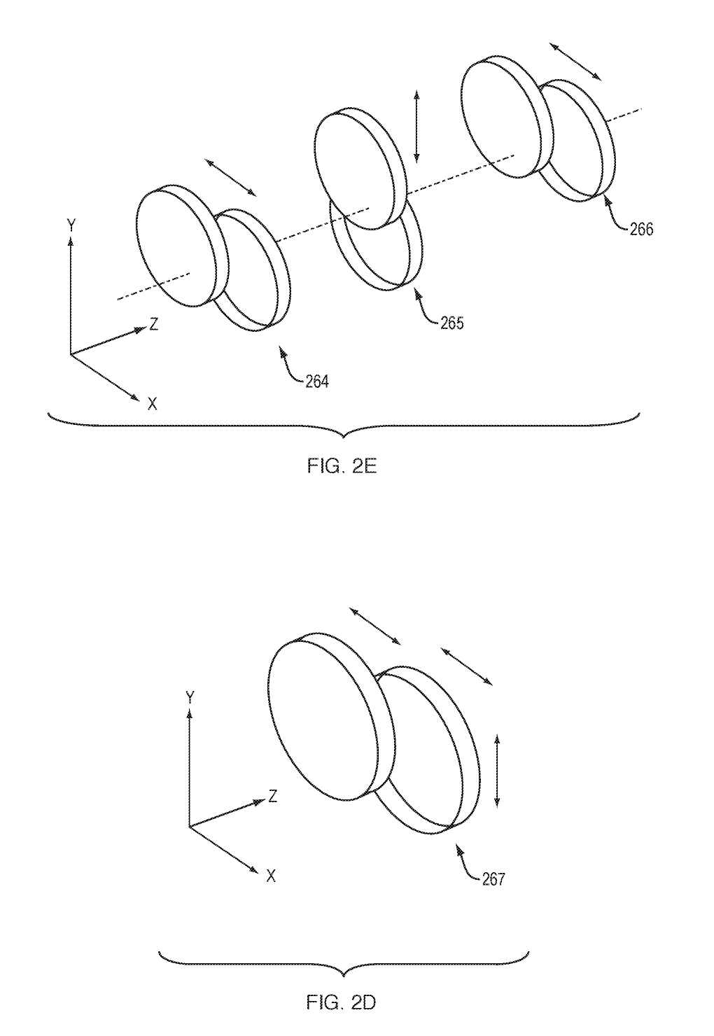

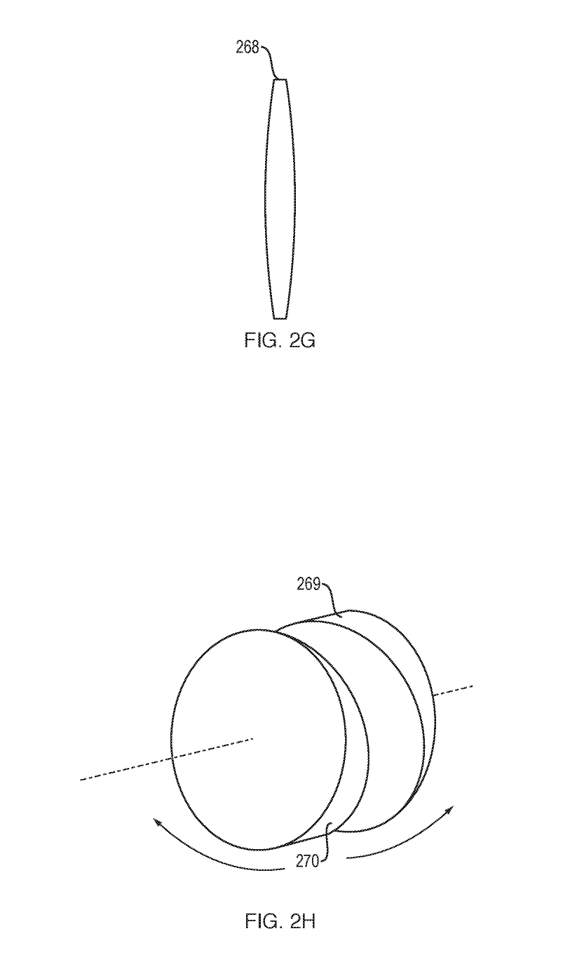

FIGS. 2A-2G show examples of variable lenses, in illustrative implementations of this invention. FIGS. 2A, 2B and 2C show Alvarez lenses 261, 262 in three different positions. FIG. 2D shows a spherocylindrical Jackson cross-cylinder lens 263. FIG. 2E shows a Humphrey lens configuration that includes three lens pairs 264, 265, 266. FIG. 2F show a Barbero-Rubenstein lens configuration that includes only one lens pair 267. In FIGS. 2E, 2F, and 2H, arrows indicate a direction in which lens in each lens pair move relative to each other. FIG. 2G shows a liquid lens 268.

In some designs, the hardware is configured for the easy replacement of a variable lens by a bigger non-variable lens with the final measured prescription to create the best viewing experience.

FIG. 2H shows a pair of Risley prisms 269, 270 for deflecting a light beam, in order to compensate for differences in inter-pupillary distance (PD).

In illustrative implementations, the refractive measurement tool includes one or more I/O devices (e.g., buttons, dials, sliders, switches, touch sensors) that detect human input. In some cases, these I/O devices (a) detect human input regarding whether an image appears clearer in a current trial or in the preceding trial, or (b) transmit motion that mechanically alters the parameters of the VLS. For example, in some cases, a human turns a dial that mechanically imparts motion to a gear assembly to cause two Alvarez lenses to slide past each other. Alternatively or in addition, one or more I/O devices in the MCD are used to detect human input.

The refractive measurement tool includes I/O devices (e.g., buttons, dials, sliders, switches, pressure sensors, other touch sensors, or potentiometers) that detect human input that involves the user directly touching, pressing on, or mechanically manipulating, the tool. In some cases, these I/O devices include other user interfaces that operate without being touched by the user, such as a capacitive sensor 230, microphone, speaker, or camera.

In illustrative implementations, the I/O devices onboard the refractive measurement tool are used to detect commands from a human user or to gather data from a human user. In some cases, the I/O devices gather subjective reports from a user during testing of an eye (such as input regarding whether a currently displayed image is clearer than a previously displayed image). In some cases, a user inputs instructions that control the refractive attributes of the VLS to adjust for the best view. For example, in some use scenarios, the user inputs an instruction that increases or decreases the spherical power of the VLS, or rotates the cylindrical axis of the VLS, or changes the cylindrical power of the VLS. In some implementations, a speaker onboard the tool outputs questions regarding a user's visual quality after an optical correction is applied, and a microphone onboard the tool records the user's verbal answers.

This ability to accept input via the refractive measurement tool is advantageous when input via the MCD is not available. For example, this capability is advantageous when all or a portion of the MCD's display screens are being used for another function (such as testing for optical aberrations of a human eye or cataracts) and are not available as a graphical user interface.

In some implementations, a human uses I/O devices (e.g., onboard the tool or the MCD) to provide feedback regarding the user's visual perception during the vision testing. For example, in some cases, a user turns a knob to select the test image that appears clearest.

In some implementations, an I/O device onboard the refractive measurement tool performs different input functions at different times. For example, in some cases, a single dial or knob is used at some times to adjust cylindrical power and at other times to adjust cylindrical axis. In some implementations, a separate I/O device onboard the tool is used to alter (e.g., toggle) input functions of other I/O devices onboard the tool (e.g., to toggle functionality of a dial, between adjustments of cylindrical power or cylindrical axis).

Alternatively, ordinary user interfaces of the MCD (or motion sensors onboard the tool or MCD) accept user feedback.

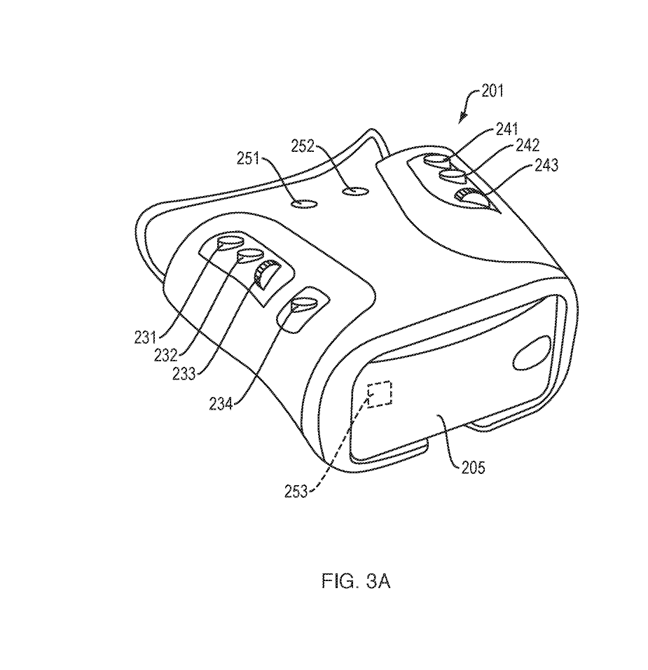

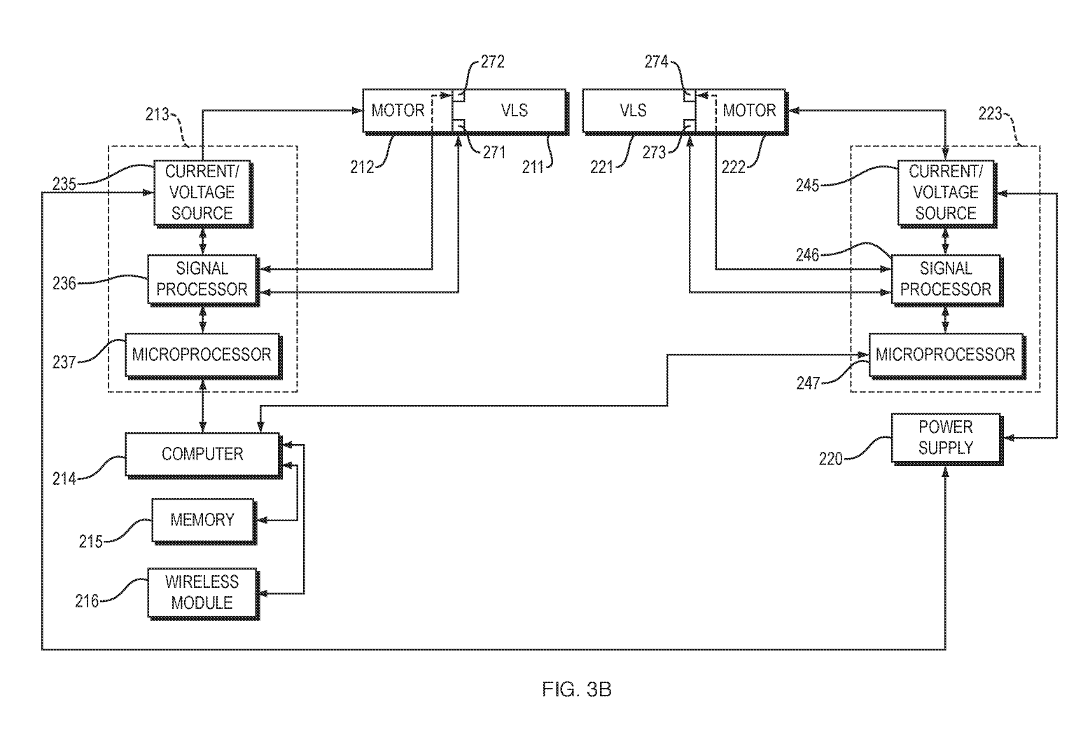

FIG. 3A shows I/O devices of a refractive measurement tool 201, in an illustrative implementation of this invention. The I/O devices include buttons 231, 232, 241, 242 and dials 233, 243 for accepting input from a user. Speaker 251 outputs audible instructions (e.g., regarding how to use the refractive measurement tool), questions (e.g., regarding whether an image appears clearer after the most recent change in VLS setting) or information (e.g., regarding the user's eyeglass prescription). Microphone 252 records audible input from a user, including information about the user (such as a user's age, gender or most recent eyeglass prescription) and subjective reports regarding visual perceptions of a user (e.g., in response to a question that is audibly outputted by the speaker). In addition, in some cases, one or more sensors (e.g., 253) onboard an MCD 205 attached to the tool 201 function as I/O devices for accepting input from, and producing output to, a human user.

FIG. 3B is a block diagram that shows hardware components of a refractive measurement tool, in an illustrative implementation of this invention. In the example shown in FIG. 3B, the right electronics module (EM) 213 includes a current source or voltage source 235, a signal processor 236 and microprocessor 237. Similarly, the left electronics module (EM) 223 includes a current source or voltage source 245, a signal processor 246 and microprocessor 247. For example, in some cases, each EM controls current or voltage applied to an actuator (e.g., motors 212, 222). For example, in some cases, each current or voltage source (e.g., 235, 245) comprises a programmable DC voltage source or programmable DC current source that provides current or voltage to a motor (e.g., 212, 222). Sensors 271, 273 detect position, displacement or other data that provides feedback regarding the operation of actuators (e.g., motors 212, 222). Motors 212, 222 actuate motion that adjusts a refractive attribute of a variable lens (e.g., 211, 221) in a VLS. For example, in some cases, the motors rotate or translate one or lenses in a variable lens relative to each other. Alternatively or in addition, motors 212, 222 translate or rotate binocular components of the refractive measurement tool relative to each other, in order to adjust PD. In some cases: (a) the VLS 211, 221 includes liquid lenses; and (b) the current/voltage sources 235, 245 each comprise a programmable DC voltage supply for applying a variable voltage to a liquid lens.

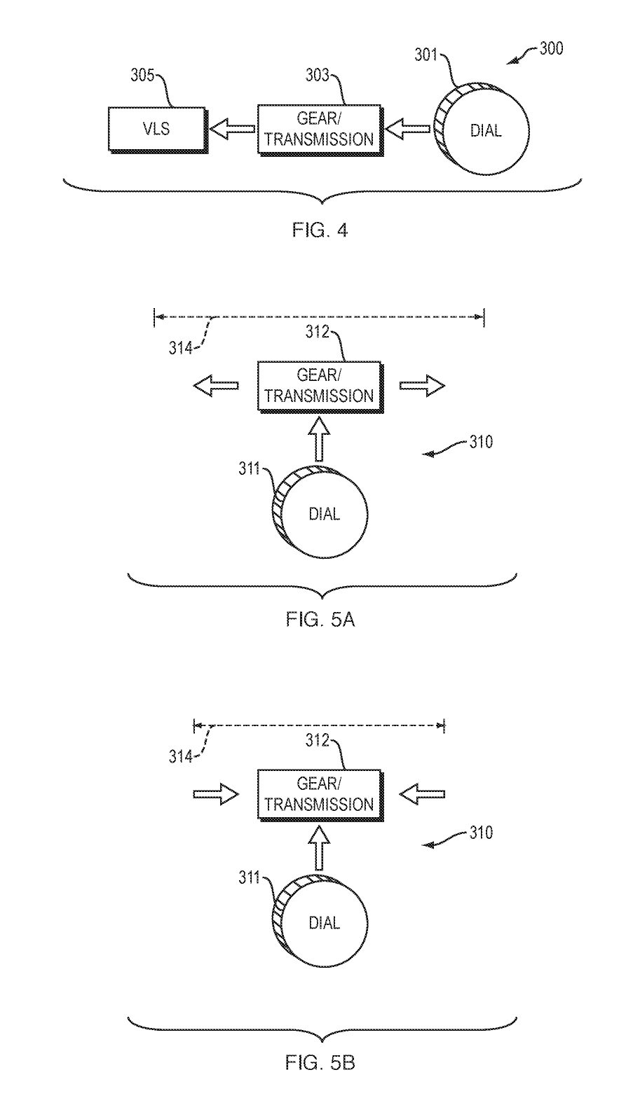

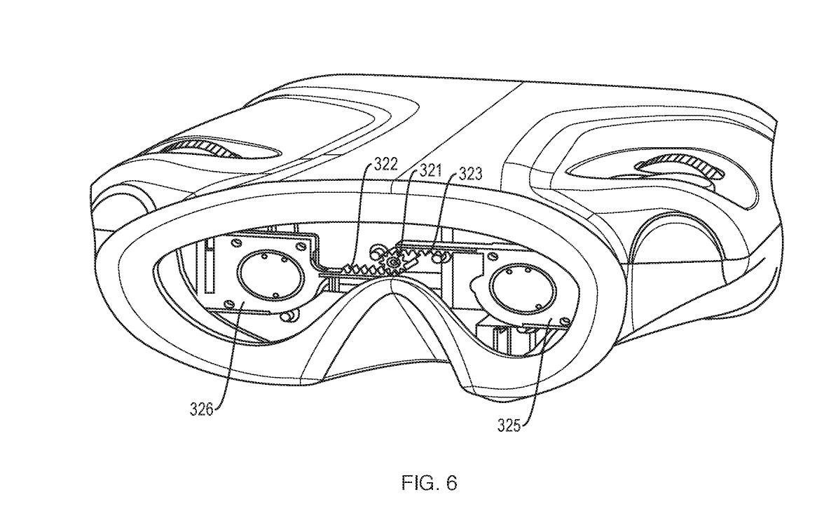

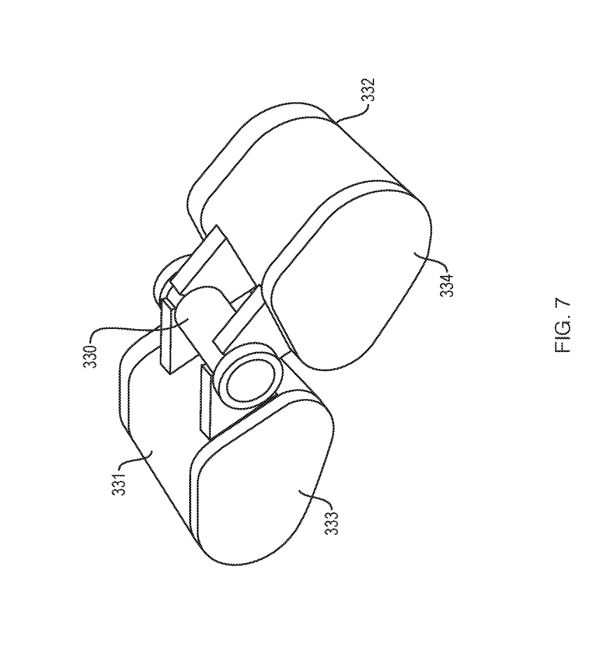

FIGS. 4, 5A and 5B each show an actuator that is powered by a user imparting mechanical motion to an I/O device, in illustrative implementations of this invention.

In FIG. 4, an actuator 300 actuates motion (e.g., translation or rotation) of lenses in a variable lens system relative to each other. A user turns a dial 301, which in turn imparts mechanical force to a system for transmitting mechanical force (e.g., a transmission or gear system) 303, which in turn transmits mechanical force to lenses in a variable lens in a VLS 305.

In illustrative implementations, it is desirable that the eye being tested and the lenses used for the test are optically aligned, in order to avoid wedging effects (shift in the perceived image) and prismatic errors. If not aligned, the user may see through a different optical power and may experience double vision when looking inside the device.

In illustrative implementations, the refractive measurement tool is bi- ocular and includes sensors (e.g., 272, 274) for measuring inter-pupillary distance. In these implementations, the inter-pupillary distance is adjustable. In some cases, the inter-pupillary distance is adjustable by mechanical motion imparted by the user. For example, in some cases the user turns a knob or dial to adjust inter-pupillary distance. In other cases, the user applies mechanical pressure to swivel the right and left portions of a bi-ocular tool about a joint to adjust the inter-pupillary distance. In other cases, the user uses an I/O device (e.g., onboard the tool) to input an instruction to adjust the inter-pupillary distance, and one or more actuators then adjust the inter-pupillary distance. One or more sensors (e.g., 272, 274) detect the new inter-pupillary distance. In some cases, a camera is used to measure changes in inter-pupillary distance.

FIGS. 5A and 5B show an actuator for adjusting PD of a binocular refractive measurement tool. In FIG. 5A, the actuator 310 is increasing PD 314. In FIG. 5B, the actuator 310 is reducing PD 314. A user turns a dial 311, which in turn imparts mechanical force to a system for transmitting mechanical force (e.g., a transmission or gear system) 312, which in turn transmits mechanical force to adjust PD.

In some cases, a rack and pinion is used to adjust the inter-pupillary distance, and a linear potentiometer is used to gather data indicative of inter-pupillary distance.

FIG. 6 shows a rack and pinion actuator for adjusting PD, in an illustrative implementation of this invention. This rack 322, 323, and pinion 321 mechanism adjusts the position of the left and right lenses simultaneously in order to match the distance between the user's eyes. In the example shown in FIG. 2C, the central gear (pinion) 321 is controlled by a dial that is turned by a user. In some cases, rotation of the dial imparts mechanical force that is transmitted to the pinion 321. In other cases, rotation of the dial controls a motor that rotates the pinion 321.

FIG. 7 shows a swivel joint for adjusting PD, in an illustrative implementation of this invention. In the example shown in FIG. 7, the refractive measurement tool is bi-ocular, with two ocular components 331, 332. These two components 331, 332 are configured to rotate about a swivel joint 330, when a user applies mechanical force to the components 331, 332. This rotation about joint 330 adjusts the PD. MCDs 333 and 334 are releasably attachable to ocular components 331 and 332, respectively.

In alternative implementations, the tool is monocular. In that case, the variable lens system (VLS) includes a lens system for only one eye. In the monocular case, the tool includes a viewport or window, configured such that a single eye looks through the tool. FIG. 8 shows a monocular refractive measurement tool 800, in an illustrative implementation of this invention. In the example shown in FIG. 8, an MCD 802 is attached to one end of the tool 800, and the tool has a single eyepiece 801.

In many embodiments of this invention, a user looks through the lenses to the MCD screen. However, in some scenarios, the user later performs additional tests, in which the user removes the MCD, and then uses the tool (including the lenses that have been adjusted) to check how the applied corrections work for real world scenes.

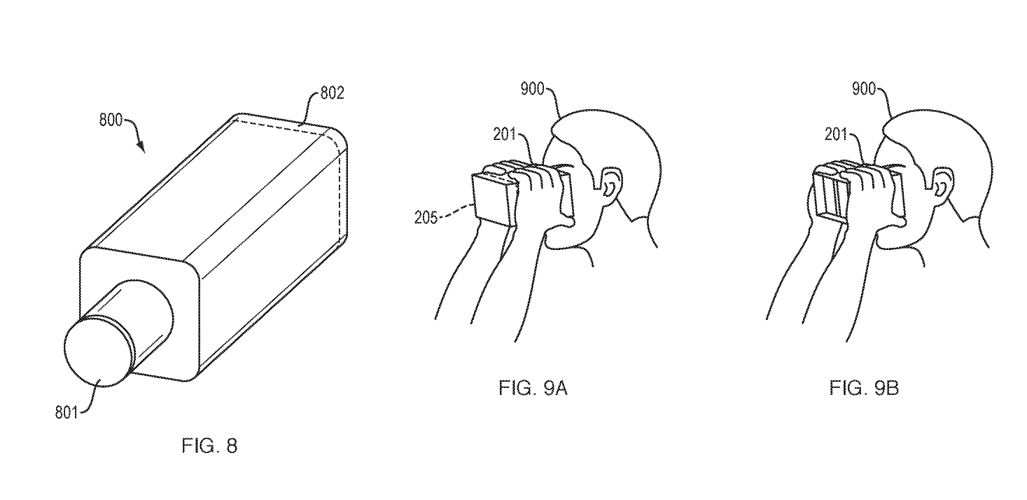

FIG. 9A shows a user 900 holding a refractive measurement tool 201 up to his eyes, in an illustrative implementation of this invention. An MCD 205 is attached to the tool 201.

FIG. 9B shows a user 900 holding a refractive measurement tool 201 up to his eyes, without an MCD attached to the tool, in an illustrative implementation of this invention. In the example shown in FIG. 9B, a vision validation test is performed after the MCD has been removed from the front of the tool. Instead of looking at the MCD screen, the user looks at a distant object (e.g. an eye chart). In this case, the tool is connected with the MCD via a wireless interface (e.g., 216). In some use scenarios, a refractive measurement tool with the MCD absent (as in the example shown in FIG. 9B) is used to determine a refractive correction while the user is engaged in an ordinary activity, such as watching television or reading a book.

In some implementations, the spherical power of the VLS is set according to the distance from the VLS to the MCD screen. Specifically, to create a refractive correction X in diopters, the optical power in diopters of the VLS is set to: E=X+1/L, where L is the distance in meters from the VLS to the scene being imaged (which scene is the MCD screen, if the MCD is attached to the front of the tool). When the MCD is removed from the front of the tool and the user is looking at distant real world scenes (e.g., FIG. 9B), L becomes infinity and thus the second term on the right hand side of the above equation may be removed.

In some implementations, refractive assessments are produced for both eyes at the same time or for one eye at a time.

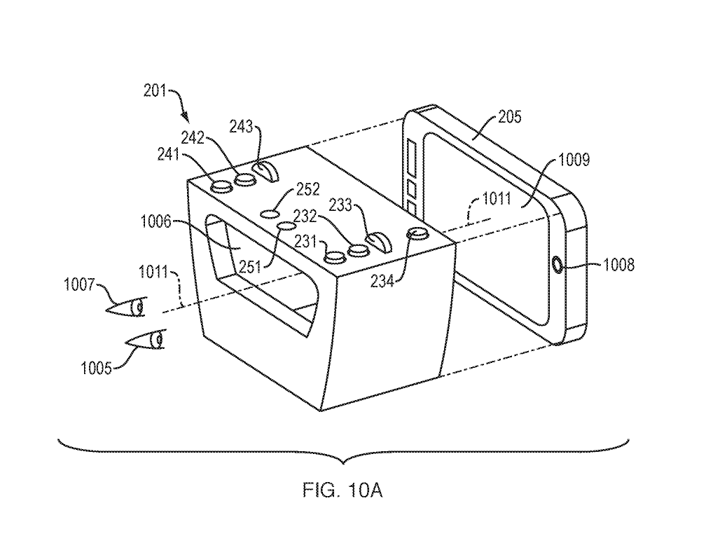

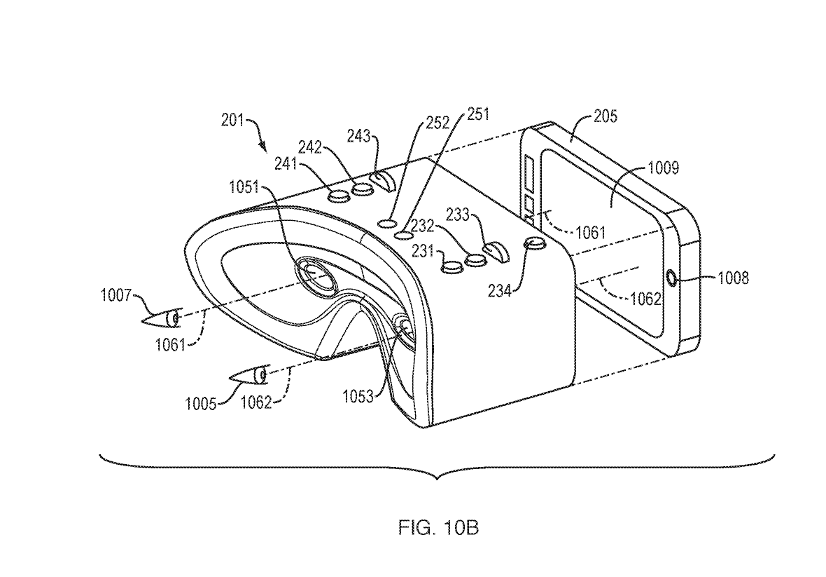

FIGS. 10A and 10B are each an exploded view of a refractive measurement tool 201, in an illustrative implementation of this invention. In FIG. 10A, the tool has a single viewport for both of the user's eyes. In FIG. 10B, the tool has two holes, one hole for each of the user's eyes. The refractive measurement tool 201 includes a housing. In addition, the tool 201 also includes user interfaces that the user manipulates. For example, in some cases, the user interfaces include buttons 231, 232, 241, 242 and dials 233, 243.

The refractive measurement tool 201 also includes an attachment mechanism that (a) easily attaches an MCD 205 to the tool, and (b) easily releases the MCD 205 from the tool. The refractive measurement tool 201 is, over the course of its useful life, repeatedly attached to, and then detached from, an MCD 205. During times when the MCD 205 is attached to the tool 201 via the attachment mechanism, the position of the tool 201 relative to the MCD 205 is fixed. The refractive measurement tool 201 includes a window 1006 through which a user views a display screen 1009 of the MCD, when the tool 201 and MCD 205 are attached to each other.

In the exploded views of FIGS. 10A and 10B, refractive measurement tool 201 and MCD 205 appear to be separated from each other. However, in actuality, when controller 201 and MCD 205 are attached to each other, MCD 205 is touching tool 201.

In FIG. 10A, an opening or hole 1006 passes through the tool 201. A line-of-sight 1011 passes through the opening 1006 and extends to a screen 1009 of the MCD, when the MCD 205 and tool 201 are attached to each other.

In FIG. 10B, the user's right eye 1005 looks through hole 1053, and the user's left eye 1007 looks though hole 1051. Lines-of-sight 1061, 1062 pass through the holes 1051, 1053, and extend to a screen 1009 of the MCD 205, when the MCD 205 and tool 201 are attached to each other.

Thus, in FIGS. 10A and 10B, a view extends through the tool 201 such that at least a portion of a screen 1009 of the MCD 205 is visible from where eyes 1005, 1007 of a human are located, when the MCD and tool are attached to each other and a surface of the tool is pressed against the forehead and cheeks of a human user. The MCD 205 includes a camera 1008.

Depending on the particular implementation, a variety of different attachment mechanisms are used to releasably join the tool 201 and MCD 205 together. For example, in some cases, an attachment mechanism that is part of the refractive measurement tool 201 comprises: (1) a clip that clips over the MCD; (2) one or more flexible bands or tabs that press against the MCD; (3) retention features that restrain the MCD on at least two edges or corners of the MCD (including retention features that are part of an opening in the tool 201); (4) a slot, opening or other indentation into which the MCD is wholly or partially inserted; (5) a socket into which the MCD is partially or wholly inserted into the tool 201; (6) a door or flap that is opened and closed via a hinge, which door or flap covers a socket or indentation into which the MCD is inserted; (7) a mechanism that restrains motion of the MCD, relative to the tool 201, in one or more directions but not in other directions; (8) a mechanism (e.g., a “snap-fit”) that snaps or bends into a position that tends to restrain motion of the MCD relative to the tool; or (9) one or more components that press against MCD and thereby increase friction and tend to restrain motion of the MCD relative to the tool 201.

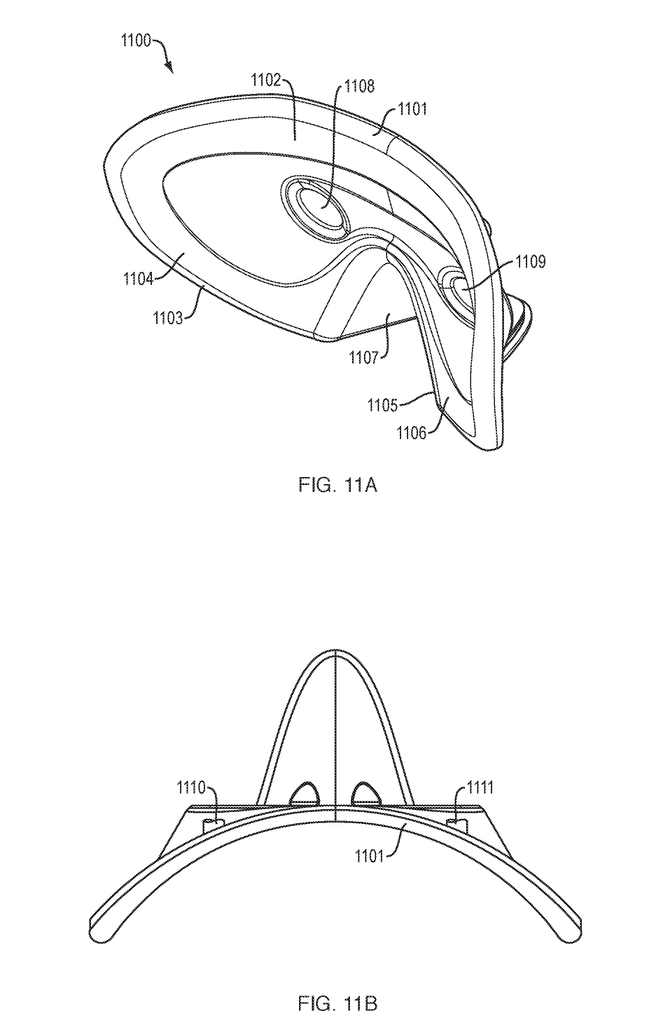

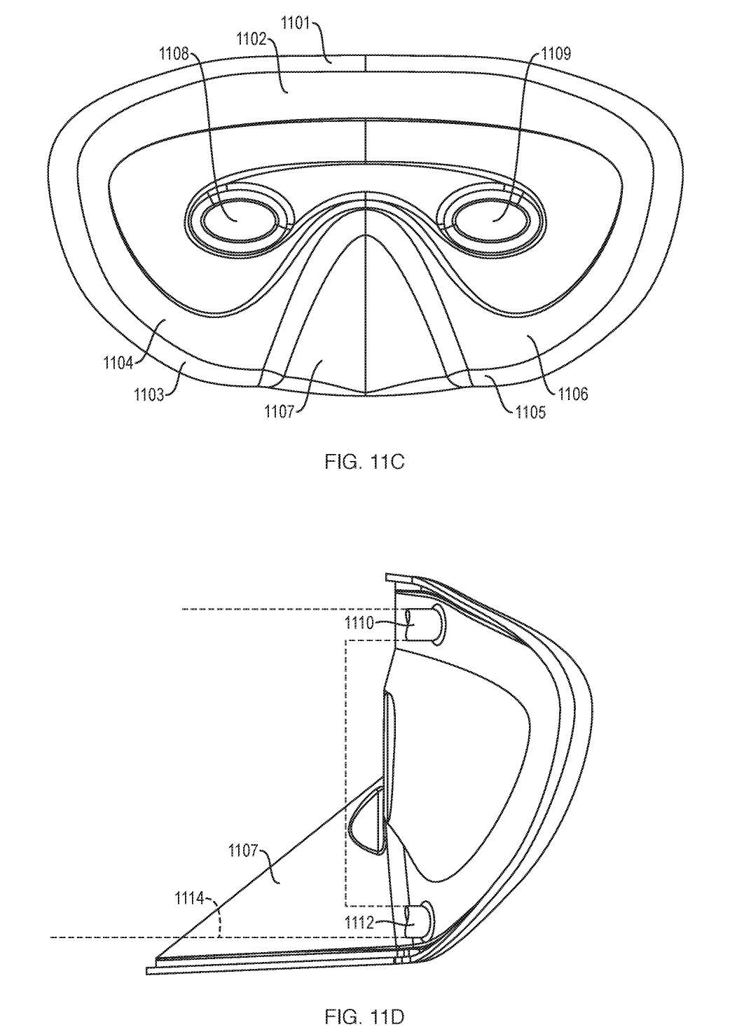

FIGS. 11A, 11B, 11C and 11D are four views of a face-fitting portion of a refractive measurement tool 201, in an illustrative implementation of this invention. The face-fitting portion is configured to be pressed against at least the forehead and cheeks of a human user. FIG. 11A is a perspective view; FIG. 11B is a top view; FIG. 11C is a back view (from the vantage point of the human user's face); and FIG. 11D is a side view.

Face-fitting portion 1100 of the tool 201 forms a surface that includes multiple curved or planar regions. Regions 1101, 1102 are configured to be pressed against (and to fit snugly against, and to conform to the shape of) the forehead of a human, either at or above the brow ridges of the human. Regions 1103, 1104 are configured to be pressed against (and to fit snugly against, and to conform to the shape of) a cheek of a human. Regions 1105, 1106 are configured to be pressed against (and to fit snugly against, and to conform to the shape of) another cheek of the human. Region 1107 is configured to be pressed against (and to fit snugly against, and to conform to the shape of) the nose of the human. Eyeholes 1108 and 1109 are holes through which a human user looks, when portion 1100 is pressed against the face of the user. Structural posts (e.g., 1110, 1111, 1112) connect the face-fitting portion 1100 to the remainder of the tool. In FIG. 11D, a portion of the main body 1114 of the tool is indicated by dashed lines.

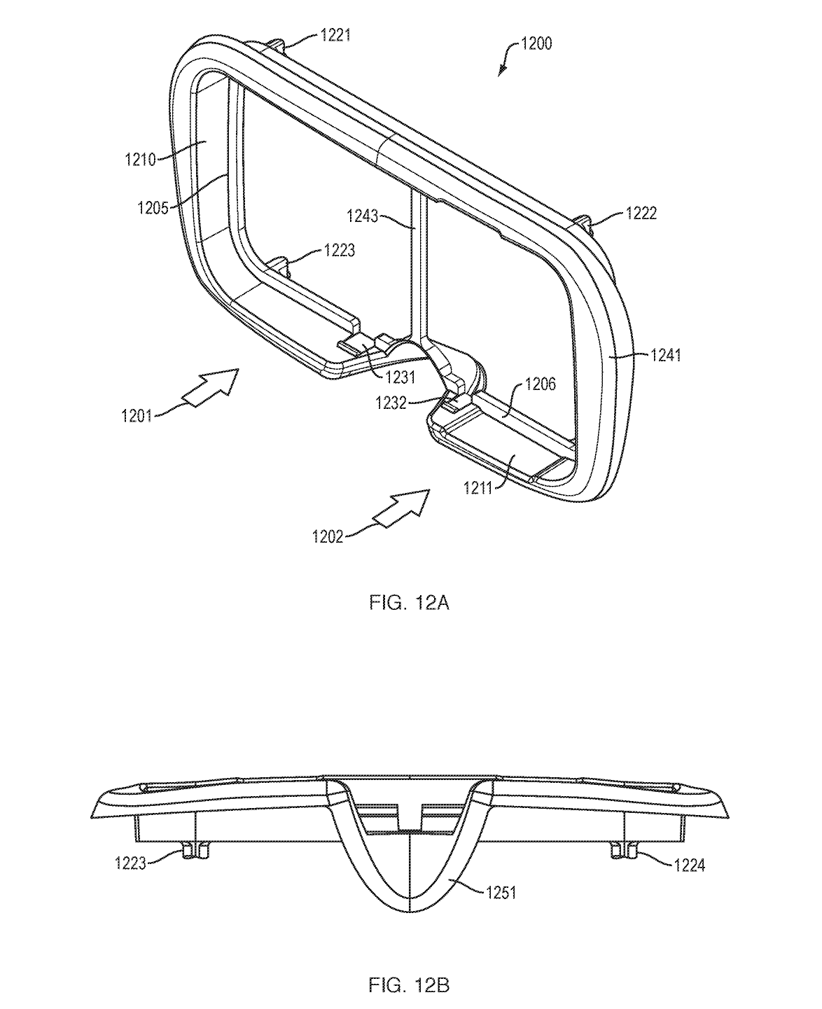

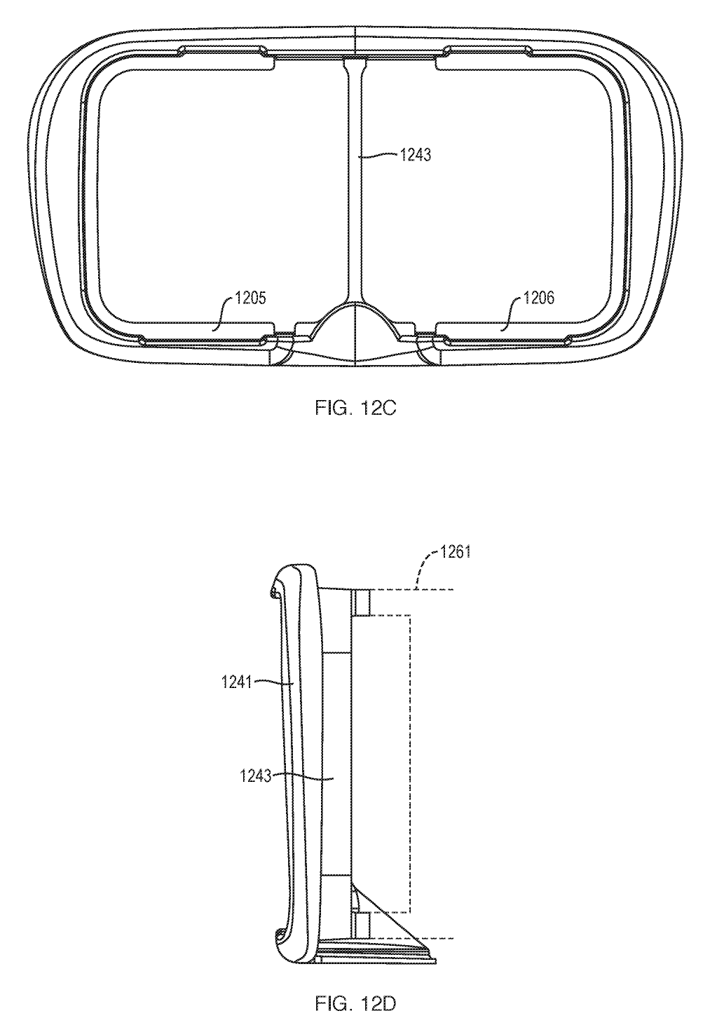

FIGS. 12A, 12B, 12C and 12D are four views of an attachment mechanism for attaching a refractive measurement tool 201 to an MCD 205, in an illustrative implementation of this invention. FIG. 12A is a perspective view; FIG. 12B is a bottom view; FIG. 12C is a back view (from the vantage point of the human user's face); and FIG. 12D is a side view.

In the example shown in FIGS. 12A, 12B, 12C and 12D, the refractive measurement tool 201 is easily attached to and easily released from the MCD 205. An opening in the attachment mechanism is surrounded by inner walls (e.g., wall 1210). The MCD is inserted into this opening in an insertion direction indicated by arrows 1201, 1202. The movement of MCD in the insertion direction is restrained by lips 1205, 1206. Tabs 1231, 1232 are flexible and press against the MCD when the MCD is touching lips 1205, 1206, tending to restrain movement of the MCD. The MCD is easily removed (released) from the attachment mechanism by pulling the MCD in a direction opposite to the insertion direction. A gentle pull on the MCD overcomes friction caused by the pressure exerted by tabs 1231, 1232 against the MCD. The indentation at 1211 creates a space such that user interfaces of the MCD do not press against the inner walls of the attachment mechanism when the MCD is inserted or removed from the attachment mechanism. This allows the MCD to be inserted and removed without inadvertently actuating these MCD user interfaces. The walls of the opening have an exterior surface, including region 1241. A support post 1243 connects two sides of the opening of the attachment mechanism, but is positioned such that it does not block the insertion and removal of the MCD. Region 1251 exposes part of the MCD to allow easier insertion of the MCD into the tool 201, or to allow easier removal of the MCD from the tool 201. For example, to remove the MCD, a user presses a thumb or other finger into the opening created by region 1251, presses the thumb or other finger against the MCD, and applies force to the MCD. Structural posts (including 1221, 1222, 1223, 1224) connect the attachment mechanism 1200 to the remainder of the tool 201. In FIG. 12D, a portion of the main body of 1261 of the tool 201 is indicated by dashed lines.

Depending on the particular implementation, the refractive measurement tool varies in size and shape. If the tool is handheld, it preferably remains an appropriate size and weight such that the user easily holds it up to his or her eyes for long periods of time.

This invention is not limited to the handheld bi-ocular device shown in FIGS. 1 and 9; but may be implemented in other ways, including with other form factors. Alternative form factors of the refractive measurement tool include (a) a monocular device, (b) open-view binoculars, (c) head-strapped binoculars or (d) or a lightweight device that is attached to, or an extension of, an eyeglasses frame and that is well-suited for extended use during ordinary activities, after the testing procedure to set refractive attributes of the VLS is completed.

In alternative implementations, a user does not hold the refractive measurement tool in his or her hand during vision testing. For example, in some implementations, the refractive measurement tool is a head-mounted display or is otherwise wearable. For example, in some cases: (a) headband straps hold a lightweight version (including housing and VLS) of the refractive measurement tool, positioning it in front of the user's eyes; (b) a hat, cap or helmet supports the refractive measurement tool and positions it in front of the user's eyes; or (c) eyeglass frames support a lightweight version of the tool for long-term wear during ordinary activities. Or, in some cases, the refractive measurement tool is a heavier device that is supported by a table-top or desk-top.

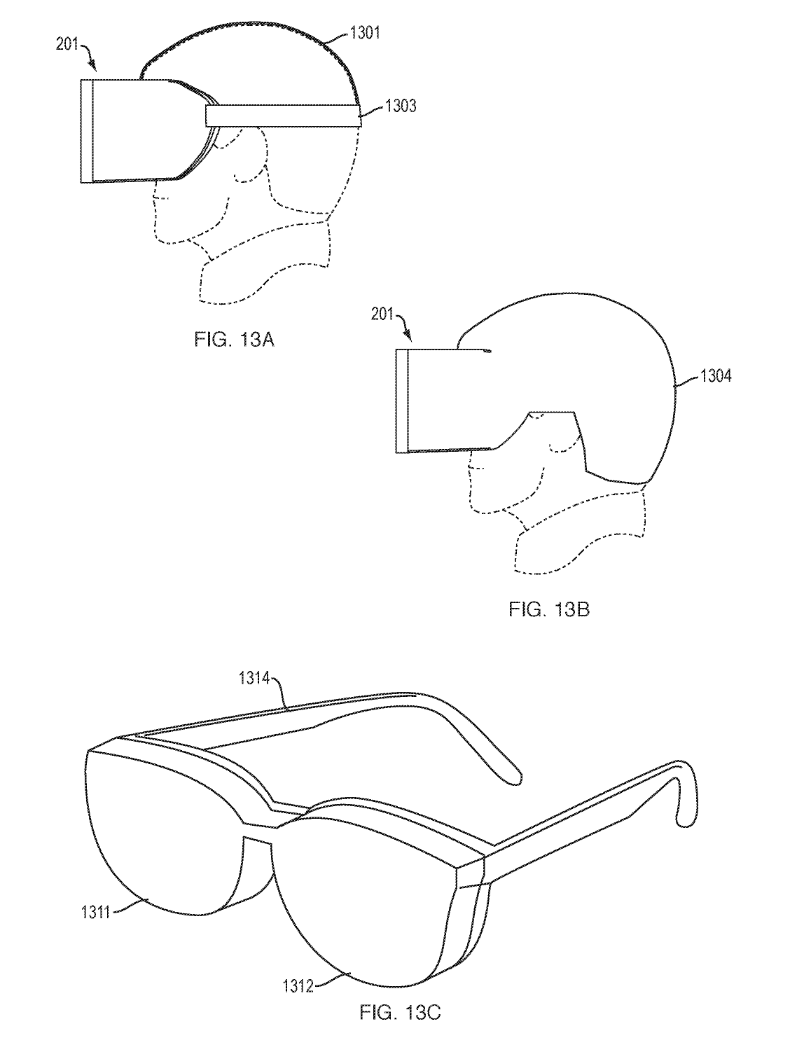

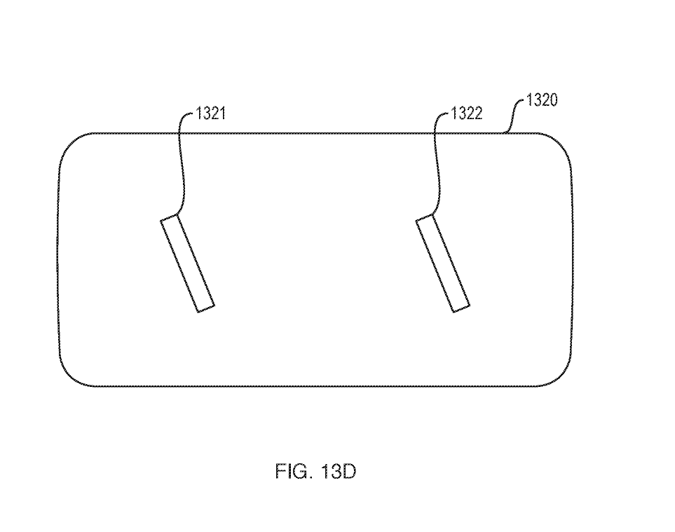

FIGS. 13A-13C show a refractive measurement tool that is attached to, or comprises part of, a head-mounted device, in illustrative implementations of this invention.

FIG. 13A shows a refractive measurement tool 201 attached to straps 1301, 1303 worn on a head, in an illustrative implementation of this invention.

FIG. 13B shows a refractive measurement tool 201 attached to headwear (e.g, a hat, cap or helmet) 1304 worn on a head, in an illustrative implementation of this invention.

FIG. 13C shows a refractive measurement tool comprising a right component 1311 and a left component 1312 attached to an eyeglass frame 1314, in an illustrative implementation of this invention.

In illustrative implementations, the user views a sequence of visual test images. In some implementations, these test images are displayed by an MCD screen. In other implementations, the MCD screen is removed from the refractive measurement tool or never attached to it, and the sequence of test images is displayed by an object in the user's environment. For example, in some cases, this object in the user's environment comprises: (a) a static letter chart or (b) a programmable electronic visual display screen that is controlled by a computer (onboard the MCD or refractive measurement tool) via a wired or wireless communication link.

In some use scenarios of this invention (including with the MCD present or absent), the vision test uses conventional test images, such as those involved in one or more of the following conventional vision acuity tests: (1) Landolt C (where the user identifies the rotation of a letter “C” as the letter becomes smaller as the test progresses); (2) an E-chart (in which a letter E is rotated in multiple sizes); (3) a Snellen chart (which comprises a sequence of letters to be read); (4) a Lea Symbol chart (where the patient identifies symbols), (5) reduction of the contrast of letters or symbols relative to a background; (6) Gabor patches (another way to measure the contrast sensitivity of the eye); or (7) an astigmatism wheel for identifying the axis of astigmatism (in which lines at different angles are shown to the user, who selects the angle that produces the clearest/sharpest/darkest image).

In some implementations, a human uses I/O devices (e.g., onboard the tool or MCD) to control test patterns displayed on the MCD screen. For example, in some cases, the input controls position or orientation of a test pattern displayed on the MCD screen (such as changing the orientation of first C to match the orientation of a second C, or aligning one pattern with another pattern). In some cases, the input controls the contrast or line thickness of a test pattern displayed on the MCD screen. In some cases, a user turns a dial or knob to look at different options (each option comprising a different visual pattern or different refractive setting of the VLS), and uses a button to confirm selection of the best image.

Depending on the particular implementation, the visual acuity test is performed in different ways. For example, in some use scenarios: (a) images are presented to only one eye at a time; (b) the same image is simultaneously presented to both eyes, or (c) different images are simultaneously presented to the right and left eyes. For example, in some use scenarios, different portions of the MCD screen are shown to the right and left eyes, to create bi-ocular effects such as parallax and 3D simulations to control and test convergence. Or, for example, in some use scenarios, a portion of the MCD screen displays test images to the eye being tested, and another portion of the MCD screen displays visual cues to the second eye, in order to control accommodation or convergence of both eyes.

FIG. 13D shows images 1321, 1322 displayed on a screen 1320, in an illustrative implementation of this invention. This invention is not limited to any particular number, size, shape, orientation or type of images. Images 1321, 1322 symbolize any images, including any number, size, shape, orientation or type of images.

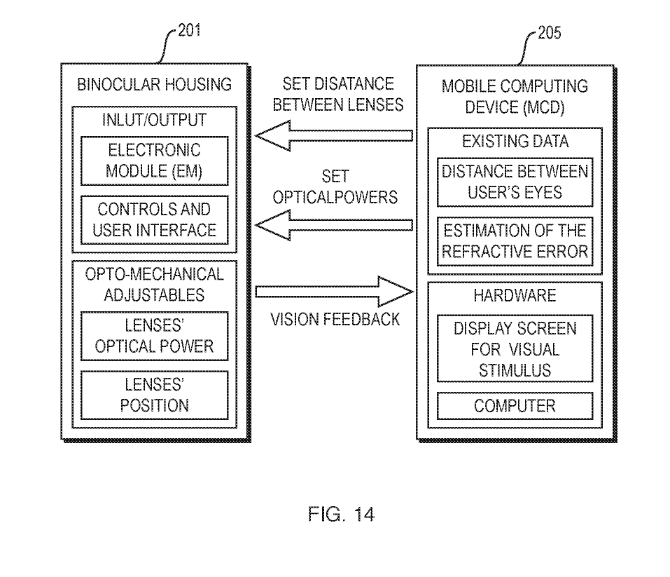

FIG. 14 is a block diagram of a refractive measurement tool 201 attached to an MCD 205, in an illustrative implementation of this invention. In the example shown in FIG. 14, a computer in the MCD (e.g., a smartphone or cell phone) sets the inter-pupillary distance and then the optical powers for the lenses. Then the user's vision performance is measured via the I/O devices and user interface in the bi-ocular tool.

### Objective Refractive Measurements

In some implementations, objective measurements of refractive error of an eye are also taken. The objective measurements do not involve input from the user regarding the user's visual perceptions. In some implementations, objective measurements are interspersed with subjective measurements (which involve the VLS), in order to validate and refine measurements of refractive aberrations of readings. For example, in some cases, a Shack-Hartmann aberrometer employs a VLS to optimize (for best focus) a captured spot diagram that reflects from the retinal layer, thus improving the accuracy of the Shack-Hartmann vision test.

In some implementations, the apparatus for objective refractive measurement comprises one or more of the following: (1) an auto-refractor, which performs a modified Scheiner's test with a lens and fundus camera to assess the image quality of a known source falling into the retina; (2) a Shack-Hartmann device for wavefront sensing, which analyzes the distortions of a known light pattern reflected onto a human retina and creates a wavefront map; (3) a retroillumination system, which captures images of an eye structure while illuminating the eye structure from the rear (e.g., by reflected light), (4) apparatus for retinoscopy (in which deformation of a slit reflected into a retina is analyzed as corrective lenses are added), and (5) a Tscherning aberrometer.

In some implementations, this invention comprises a device for optimizing a high-order wavefront map of an eye. An additional apparatus takes objective measurements to determine a wavefront map of refractive aberrations of an eye. Results from iterative VLS subjective measurements are used to (a) to improve accuracy of the wavefront map; (b) to determine a conversion from the high-order map to a low-order, spherocylindrical aberration correction, which conversion optimizes for visual acuity; or (c) to achieve a maximum eye relaxation that yields an acceptable visual acuity. For example, in some cases, this additional apparatus (a) includes one or light sources, masks, light sensors and computers, and (b) is housed onboard the refractive measurement tool or the MCD.

In some implementations of this invention, the objective measurements comprise EyeNetra Photorefraction Measurements (EPMs), and the additional apparatus includes apparatus for taking EPMs.

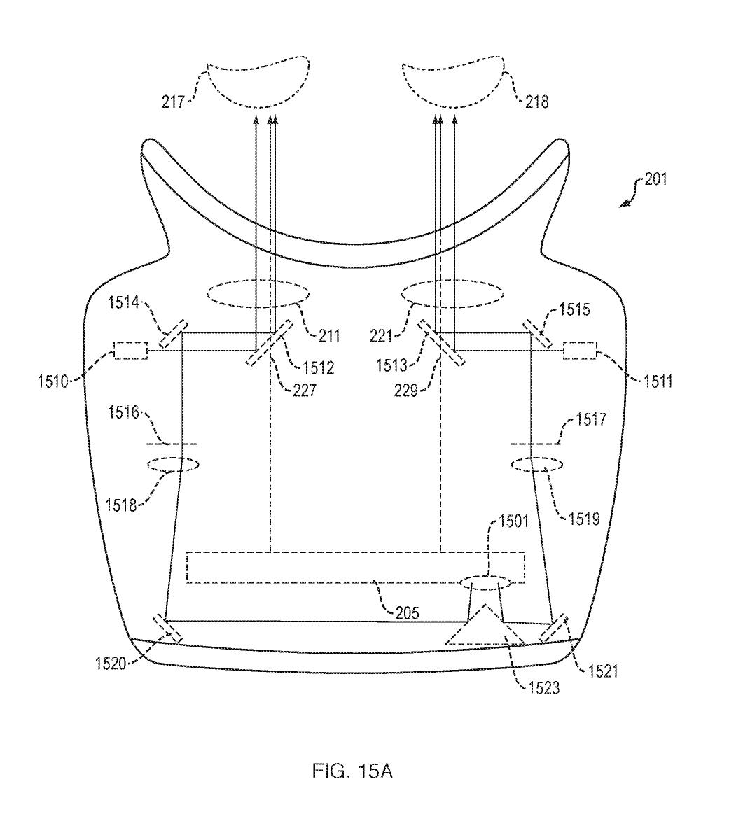

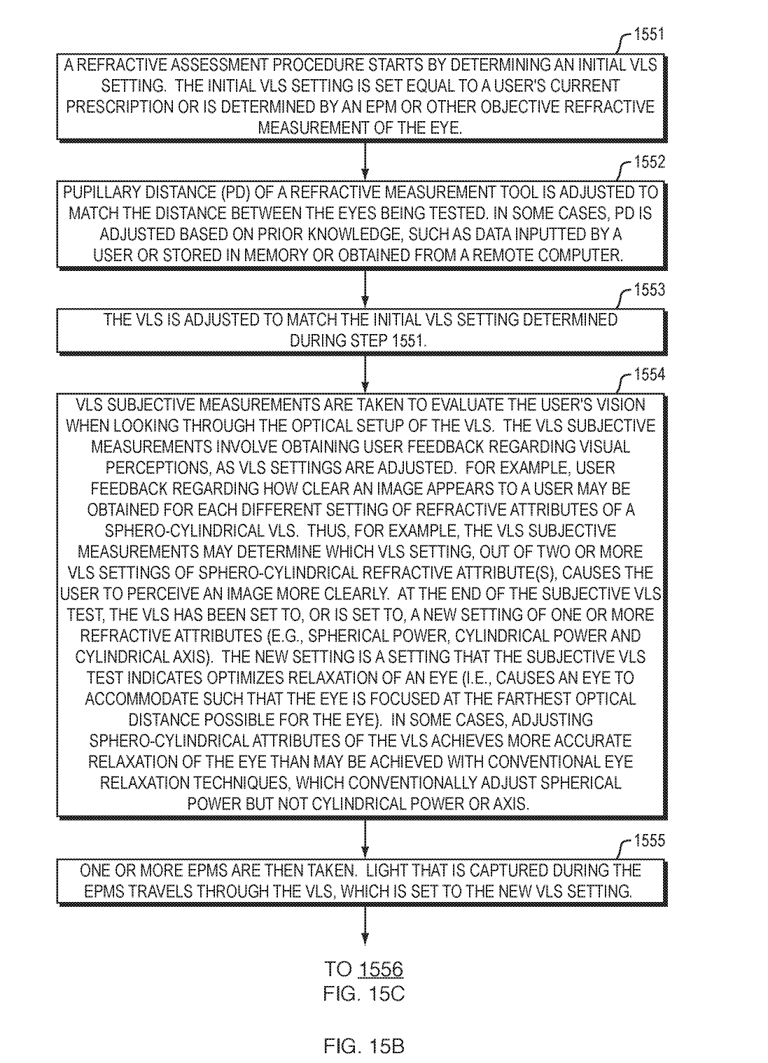

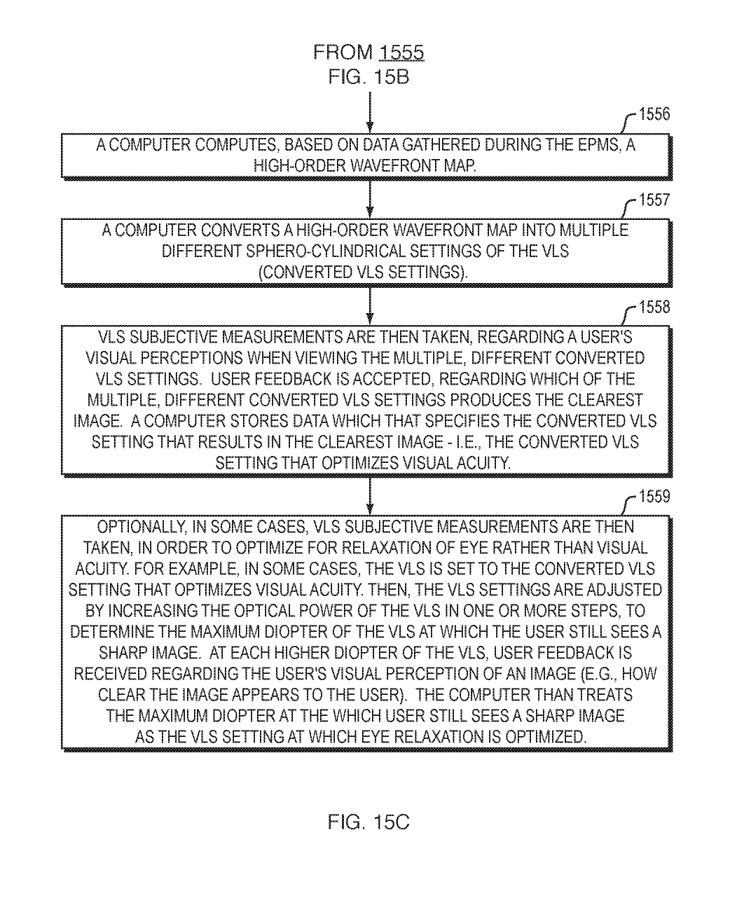

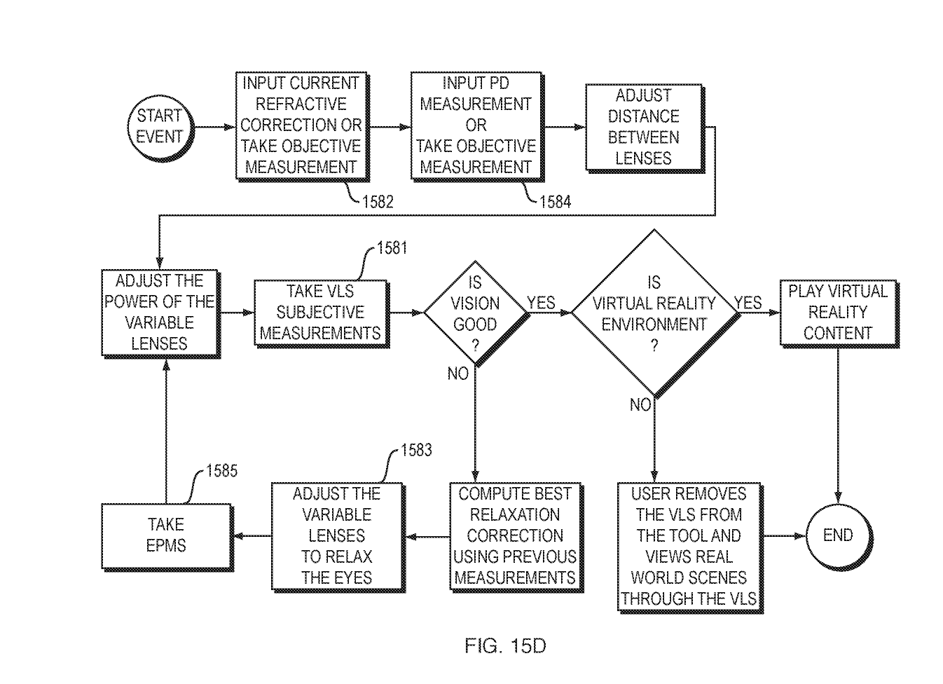

FIG. 15A shows a refractive assessment tool 201 that includes a variable lens system and apparatus for taking EPMs, in an illustrative implementation of this invention. In the example shown in FIG. 15A, a MCD 205 (e.g., a smartphone) is attached to the tool 201. The MCD 205 includes a camera 1501. The camera 1501 and the display screen of the MCD 205 are on opposite sides of the MCD from each other. The camera 1501 captures images while the user looks through the adjustable lenses 211, 221 into the display screen of the MCD 205.

In the example shown in FIG. 15A: Light sources 1510, 1511 emit light. Light from light source 1510 reflects off beam splitter 1512, then travels through variable lens 211, then enters the user's right eye 217, then reflects out of the user's right eye 217, then travels through the variable lens 211, then reflects off beam splitter 1512, then reflects off mirror 1514, then travels through mask 1516, then travels through lens 1518, then reflects off mirror 1520, and then is refracted by prism 1523 such that the light strikes a first region of an image sensor of camera 1501. Likewise, light from light source 1511 reflects off beam splitter 1513, then travels through variable lens 221, then enters the user's left eye 218, then reflects out of the user's left eye 218, then travels through the variable lens 221, then reflects off beam splitter 1513, then reflects off mirror 1515, then travels through mask 1517, then travels through lens 1519, then reflects off mirror 1521, then is refracted by prism 1523 into a second region of an image sensor of camera 1501. Thus, the prism 1523 steers light from the right eye 217 into a first region of the image sensor of camera 1501, and steers light from the left eye 218 into a second region of the image sensor. (The first and second regions do not overlap). This all happens while the user looks at the display screen of the MCD 205. Light (e.g., 1524, 1525) from the display screen of the MCD 205 travels through the beamsplitters 1512, 1513 and then through the VLS 211, 221 to the user's eyes 217, 218. For example, in some cases, lenses 1518, 1519 are each an objective lens.

Alternatively, other configurations of hardware and optical elements for taking EPMs may be employed, instead of the particular example shown in FIG. 15A. Any hardware and optical configuration disclosed in the EPM Patent Application may be housed in a refractive measurement tool 201 that also houses a VLS and other hardware (such as the hardware shown in FIGS. 1, 3A and 3B hereof). For example, more than one mask (spatial modulator) may be positioned in each optical path (from a light source, into and out of an eye, and to a camera)—instead of only one mask, as shown in FIG. 15A.

FIGS. 15B and 15C show steps in a method that includes taking EPMs and taking VLS subjective measurements, in an illustrative implementation of this invention.

In the example shown in FIGS. 15B and 15C, a method includes the following steps: A refractive assessment procedure starts by determining an initial VLS setting. The initial VLS setting is set equal to a user's current prescription or is determined by an EPM or other objective refractive measurement of the eye (Step 1551). PD of a refractive measurement tool 201 is adjusted to match the distance between the eyes being tested. In some cases, PD is adjusted based on prior knowledge, such as data inputted by a user or stored in memory or obtained from a remote computer. (Step 1552). The VLS is adjusted to match the initial VLS setting determined during Step 1551 (Step 1553).

In the example shown in FIGS. 15B and 15C, VLS subjective measurements are then taken to evaluate the user's vision when looking through the optical setup of the VLS. The VLS subjective measurements involve obtaining user feedback regarding visual perceptions, as VLS settings are adjusted. For example, user feedback regarding how clear an image appears to a user may be obtained for each different setting of refractive attributes of a spherocylindrical VLS. Thus, for example, the VLS subjective measurements may determine which VLS setting, out of two or more VLS settings of spherocylindrical attribute(s), causes the user to perceive an image more clearly. At the end of the subjective VLS test, the VLS has been set to, or is set to, a new setting of one or more refractive attributes (e.g., spherical power, cylindrical power and cylindrical axis). The new setting is a setting that the subjective VLS test indicates optimizes relaxation of an eye (i.e., causes an eye to accommodate such that the eye is focused at the farthest optical distance possible for the eye). In some cases, adjusting spherocylindrical attributes of the VLS achieves more accurate relaxation of the eye than may be achieved with conventional eye relaxation techniques, which conventionally adjust spherical power but not cylindrical power or axis. (Step 1554).

In the example shown in FIGS. 15B and 15C, one or more EPMs are then taken. Light that is captured during the EPMs travels through the VLS at the new VLS setting (Step 1555). A computer computes, based on data gathered during the EPMs, a high-order wavefront map (Step 1556). A computer converts a high-order wavefront map into multiple different spherocylindrical settings of the VLS (converted VLS settings) (Step 1557). VLS subjective measurements are then taken, regarding a user's visual perceptions when viewing the multiple, different converted VLS settings. User feedback is accepted, regarding which of the multiple, different converted VLS settings produces the clearest image. A computer stores data which that specifies the converted VLS setting that results in the clearest image—i.e., the converted VLS setting that optimizes visual acuity (Step 1558).

Optionally, in some cases, VLS subjective measurements are then taken, in order to optimize for relaxation of eye rather than visual acuity. For example, in some cases, the VLS is set to the converted VLS setting that optimizes visual acuity. Then, the VLS settings are adjusted by increasing the optical power of the VLS in one or more steps, to determine the maximum diopter of the VLS at which the user still sees a sharp image. At each higher diopter of the VLS, user feedback is received regarding the user's visual perception of an image (e.g., how clear the image appears to the user). The computer than treats the maximum diopter at the which user still sees a sharp image as the VLS setting at which eye relaxation is optimized (Step 1559).

Alternatively or in addition, in some implementations, the VLS is adjusted to best focus the patterns reflected from the retina. Then EPMs are taken. The better focused images improve the accuracy of the EPMs. The improvement in image acquisition leads to better accuracy for the intermediary and final refractive aberration readings.

In some implementations, multiple EPMs are taken, each at a different setting of the VLS, in order to improve accuracy of the refractive measurements. For example, in some use scenarios, a VLS is adjusted to a new setting. The new VLS setting is at a different diopter than the prior VLS setting. During the short period of time in which an eye adjusts to the new VLS setting, multiple EPMs are taken of the eye. The EPMs stop changing when the eye finishes accommodating to the new, different diopter VLS setting. The speed at which the eye accommodates to the new VLS setting indicates how well the eye is relaxed. (In some cases in some diopter ranges, the longer it takes for the eye to accommodate to an increased diopter of the VLS, the closer the VLS is to a setting that maximizes relaxation of the eye).

In some implementations, VLS subjective measurements determine a total accommodation range of a user's eye, thereby improving the accuracy of EPM-based prescriptions for distance and reading activities. Given a final distance refraction, adjustments to the VLS may simulate closer objects. In some cases, VLS optical power is increased in increments. At each increment, VLS subjective measurements are taken. This procedure may determine the diopter at which the additional optical power places an object optically too close to the person, such that the user cannot focus on the object anymore. Based on this measured diopter, a computer determines a range of accommodation of the user and estimates the best reading glasses for activities that have distinct reading distances (driving, reading, using a computer, using a phone, etc).

In some implementations of this invention, a computer calculates a high-order Zernike curve (i.e., Zernike polynomial), based on one or more EPMs. A computer then decomposes this Zernike curve into the three main components of the Zernike curve: sphere, cylinder and axis. The computer performs different algorithms to determine multiple, different decompositions of the Zernike curve. Each of the decompositions is a spherocylindrical VLS setting that does not perfectly replicate all of the information in the higher-order Zernike curve. Each different decomposition algorithm ignores attributes of the Zernike curves. Then VLS subjective measurements are taken as the VLS is adjusted to each of these different decompositions. In these VLS subjective measurements, feedback from the user is obtained, regarding the user's visual perceptions at each of these different decompositions (spherocylindrical VLS settings). In some cases, the VLS subjective measurements determine which decomposition (spherocylindrical VLS setting) produces the best visual acuity. In other cases, the VLS subjective measurements determine which decomposition (spherocylindrical VLS setting) maximizes accommodation of an eye. Perception is subjective. Different users give different perceptual weights to attributes of the Zernike fitting. The VLS subjective measurements of a user automatically take the user's different perceptual weights into account, thereby determining the decomposition of the EPM-based wavefront map that is best suited for that user.

In some use scenarios, after the EPMs and VLS subjective measurements are completed, the user removes the VLS module from the body of the refractive measurement device and uses the VLS module as temporary eyeglasses since the VLS module is corrected for the person's vision.

FIG. 15D shows steps in another method that includes taking EPMs and taking VLS subjective measurements, in an illustrative implementation of this invention. The method shown in FIG. 15D includes initial steps, such as: (a) input a current refractive assessment or determine it by objective measurements such as EPMs (Step 1582); and (b) input PD or determine it by objective measurements (Step 1584). The method shown in FIG. 15D also includes the following steps (among others): Take VLS subjective measurements (Step 1581). Adjust the variable lenses to relax the eyes (Step 1583). Take EPMs (Step 1585). Steps 1581, 1583 and 1585 are in a loop.

Measuring refractive aberrations of an eye by a procedure that includes both VLS subjective measurements and EPMs has many practical benefits, including at least the following ten advantages in illustrative implementations of this invention:

First, eye relaxation is improved due to the adjustments for sphere, cylinder and axis during the VLS subjective measurements, thus improving the accuracy of the EPMs.

Second, in cases of uncorrected astigmatism, it may be difficult to predict if the eye is fully relaxed or not. The VLS subjective measurements may optically correct for the astigmatism, to remove that unpredictability.

Third, an EPM may be performed in a single exposure, reducing the time needed for testing. Even multiple EPMs may be captured very rapidly.

Fourth, a testing procedure that involves both VLS subjective measurements and EPMs is, apparently, more accurate than any technique that employs objective measurements alone. For example, the testing procedure is more accurate than a conventional Shack-Hartman aberrometer alone.

Fifth, a testing procedure that involves both VLS subjective measurements and EPMs is, apparently, more accurate than any other technique that employs subjective measurements alone.

Sixth, a testing procedure that includes both VLS subjective measurements and EPMs is, apparently, more accurate than a testing procedure that employs both NETRA Measurements and a VLS. Employing EPMs may improve accuracy because an EPM may measure higher-order aberrations.

Seventh, during EPMs, a display screen may be employed for relaxation of an eye and for displaying content (e.g., virtual reality content), instead of for displaying test images.

Eighth, in some use scenarios, comparing the VLS subjective measurements and the EPMs may detect other vision problems that are not merely refractive issues.

Ninth, the EPM may measure high-order aberration and a computer may decompose a two dimensional map (Zernike fittings) into its three main components (sphere, cylinder and axis) in order to provide an eyeglasses prescription. VLS subjective measurements may be taken to determine which decomposition method yields the best vision for each person. This process of determining the best decomposition may be performed rapidly. For example, a user may turn a knob to display different decompositions (spherocylindrical VLS settings) and then input which decomposition produces the sharpest image.

Tenth, the EPM raw readings may be improved by adjusting the VLS to focus the emitted pattern on the retinal instead of adjusting for the best user vision.

### VLS and NETRA

In some implementations: (a) the refractive measurement tool houses a VLS and a NETRA aberrometer, as defined herein; and (b) refractive aberration of an eye is assessed by taking VLS subjective measurements and NETRA Measurements, as defined herein. For example, a display screen of the MCD 205 may function as the display screen of the NETRA aberrometer. A computer onboard the MCD may control this display screen and, in some cases, may also control the VLS.

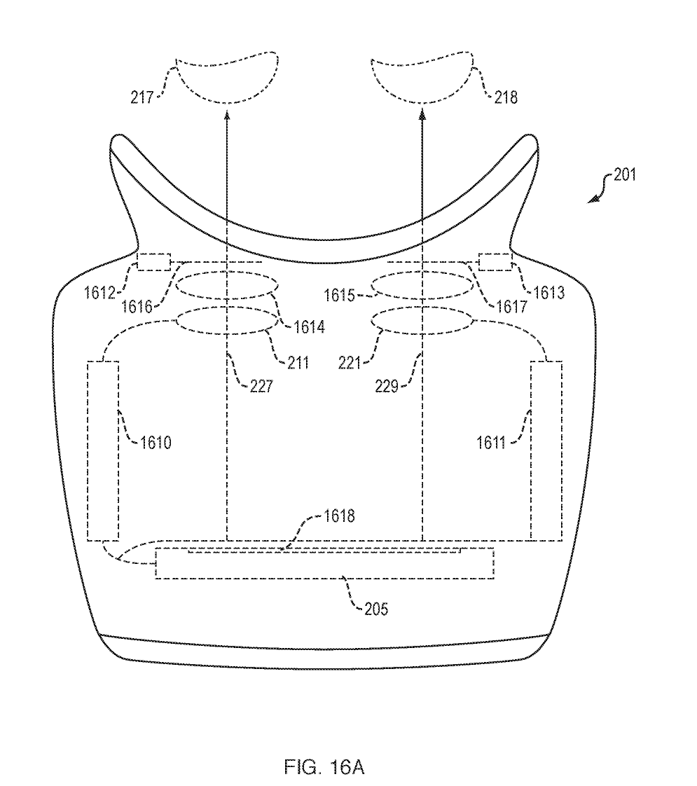

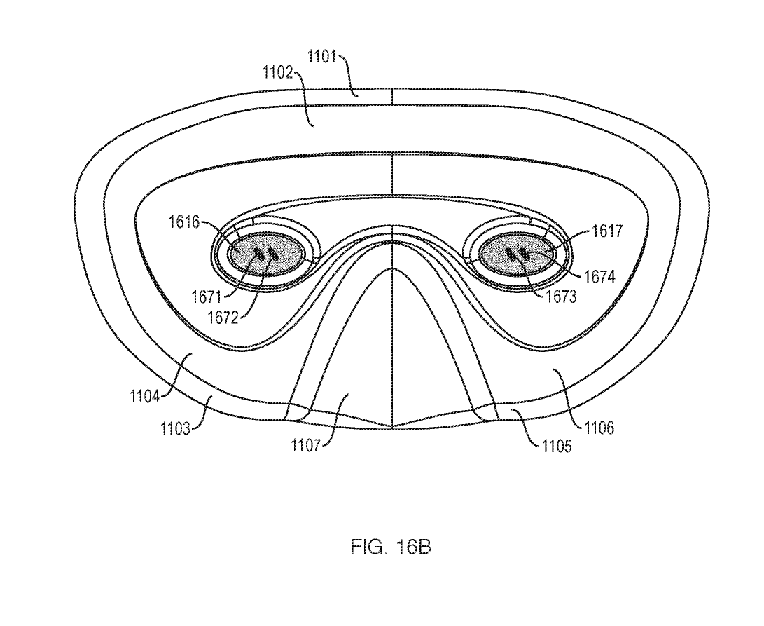

FIG. 16A shows a refractive assessment tool 201 that includes a variable lens system 211, 221 and a NETRA aberrometer, in an illustrative implementation of this invention. The NETRA aberrometer includes two masks 1616, 1617, lens for relaxing the eyes 1614, 1615 and a display screen 1618. For example, the display screen 1618 may comprise a display screen of an MCD 205 that is attached to the tool 201. The tool includes electronic circuitry 1610, 1611 for controlling the VLS and for controlling one or more actuators in the tool. For example, in some cases: (a) electronic circuitry 1610 comprises EM module 213 and motor 212, as shown in FIG. 3A; and (b) electronic circuitry 1611 comprises EM module 223 and motor 222, as shown in FIG. 3A.

In some implementations, the NETRA aberrometer includes one or more lenses (e.g., 1614, 1615) to relax the eye (accommodation) and masks 1616, 1617. For example, each mask (e.g., 1616, 1617) may filter light, such that, at any given time: (a) light from a part of the cornea that is being measured at the given time passes through the filter and (b) light from the parts of the cornea that are not being measured at the given time does not pass through the filter.

In some implementations, variable lenses in the VLS 211, 221 affect both the NETRA Measurements and relaxation of the eye, at the same time. The spherical refractive power measured at a given time is offset by the refractive power applied to the variable lens. Thus, in some implementations, an eye's optical power eye per angle is calculated in accordance with the equation: C(θ,t)=P(θ,t)−L(θ,t), where C is the final optical power, P is the measured optical correction that is measured by the NETRA aberrometer and computed in accordance with column 8, lines 25 to 35, of the NETRA Patent, L is the power applied to the lens, θ is the measuring axis and t is the time.

In some cases, each mask (e.g., 1616, 1617) in the NETRA aberrometer is a semi-transparent film that includes two parallel lines, one red and one green, placed approximately 1.2 mm apart from each other, in a blue background. The parallel lines allow light to pass through the lines. For example, if a mask is positioned such that the two lines are oriented horizontally with the red line on top of the green line, then: (a) the red line allows red light from the display screen to pass through the red line and thus through a top part of the cornea; and (b) the green line allows green light from the display screen to pass through the green line and thus thorough a bottom part of the cornea. The blue background allows blue light to pass through the whole cornea and is used mostly for the eye relaxation procedures.

FIG. 16B shows lines in masks in a NETRA aberrometer. Mask 1616 includes red line 1672 and green line 1671. Mask 1617 includes red line 1674 and green line 1673.

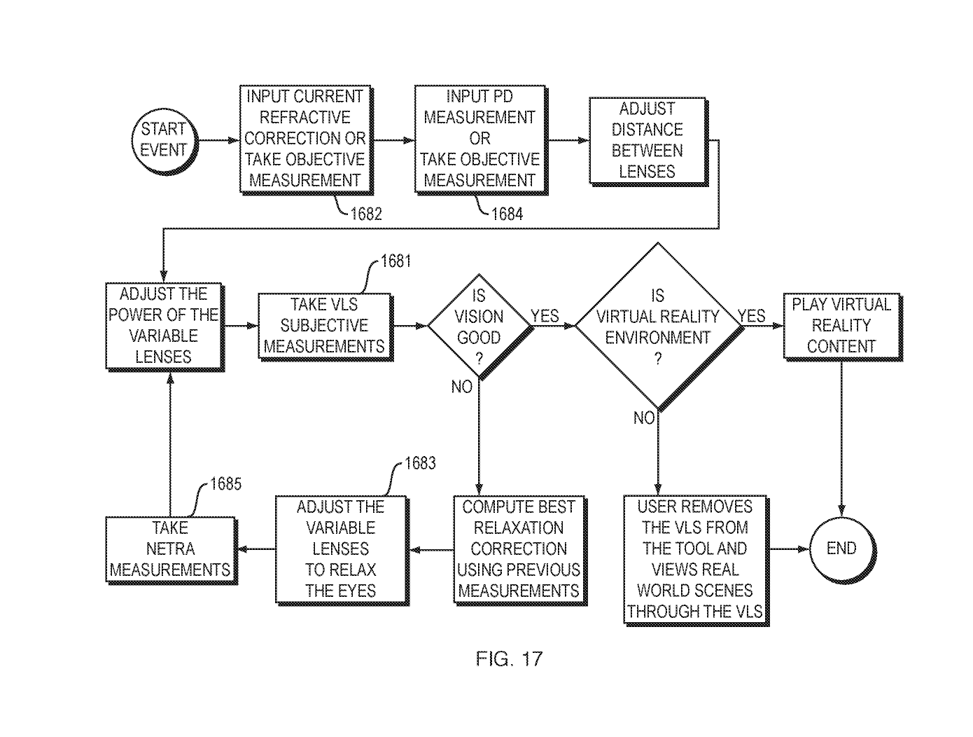

FIG. 17 shows steps in a method that includes taking NETRA Measurements and taking VLS subjective measurements. The method shown in FIG. 17 includes initial steps, such as: (a) input a current refractive assessment or determine it by objective measurements such as EPMs (Step 1682); and (b) input PD or determine it by objective measurements (Step 1684). The method shown in FIG. 15D also includes the following steps (among others): Take VLS subjective measurements (Step 1681). Adjust the variable lenses to relax the eyes (Step 1683). Take NETRA Measurements (Step 1685). Steps 1681, 1683 and 1685 are in a loop that includes other steps.

In some cases, both a VLS and a NETRA aberrometer are employed. A user looks inside the refractive measurement tool and performs a series of line alignments. One or more computers perform computations that (a) take, as an input, data regarding the alignments performed at a given time, and (b) outputs a refractive correction needed for the portion of the cornea being measured at the given time. Variable lenses in the VLS are adjusted to compensate for the optical power measured by NETRA thereby enhancing visualization of the lines for NETRA's line alignment task. This, in turn, improves accuracy and improves the relaxation of the eye. A computer performs an algorithm to best fit a sinusoidal curve to the optical powers per angle, in order to compute an eyeglasses prescription (e.g., to compute spherical power, cylindrical power and cylindrical axis of an eyeglasses prescription).

In some implementations, one or more actuators (e.g., 1612, 1613) rotate one or more of the masks (1616, 1617), such that the tool takes NETRA Measurements of optical power at different orientations of the lines. These different orientations of the lines facilitate measurements of different cylindrical axes. Thus, this ability to rotate the mask of a NETRA aberrometer is helpful for measuring for astigmatism.

Measuring refractive aberrations of an eye by a procedure that includes both VLS subjective measurements and NETRA Measurements has many practical benefits, including at least the following eight advantages in illustrative implementations of this invention:

First, eye relaxation is improved due to the adjustments for sphere, cylinder and axis during the VLS subjective measurements, thus improving the accuracy of the NETRA Measurements.

Second, in cases of uncorrected astigmatism, it is difficult to predict if the eye is fully relaxed or not. The VLS subjective measurements may optically correct for the astigmatism, to remove that unpredictability.

Third, the refractive tool, which houses both the VLS and NETRA aberrometer, may have a short optical path and small number of optical components, thus making the tool low cost in some cases. A small number of optical components is advantageous, because reducing the number of optical components tends to reduce the total optical aberrations that are created by the sum of the manufacturing optical tolerances of each component.

Fourth, a testing procedure that involves both VLS subjective measurements and NETRA Measurements is, apparently, more accurate than any technique that employs objective measurements alone or subjective measurements alone. For example, the testing procedure is more accurate than a conventional Shack-Hartman aberrometer alone.

Fifth, in some use scenarios, comparing the VLS subjective measurements and the NETRA Measurements may detect other vision problems that are not merely refractive issues.

Sixth, the VLS may compensate for vision errors while doing an alignment task. This tends to improve the accuracy of alignment tasks during the NETRA Measurements.

Seventh, the NETRA aberrometer may be implemented in an inexpensive manner, because NETRA does not require an active sensor.

Eighth, the user experience is simple. Because NETRA is also subjective, the change in user experience when changing between VLS subjective measurements and NETRA Measurements is reduced. In some cases, virtual reality content is displayed both while performing NETRA Measurements and while performing VLS subjective measurements.

In some implementations, the refractive measurement tool is used in conjunction with other subjective vision tests, such as a Scheiner disk test.

### Refractive Assessment

In some implementations, an initial refractive correction estimate and initial pupillary distance (PD) estimate are taken as inputs. The device adjusts the distance between the lenses to match to the user's PD. The lenses are adjusted to the entered refractive correction and a visual assessment is performed. If the person already has good vision, the device stops the optimization procedure. If not, the device tries another refraction and a new subjective test is performed. In some use scenarios, when a good vision is achieved (e.g., 20/20 vision), the user removes the MCD from the refractive measurement tool, in order to look at his or her surroundings with the optimized refractive correction. In some cases, a wireless connection exists between the tool and the MCD, and a computer onboard the MCD controls the VLS during additional optimization rounds to improve the refractive correction while the user looks at the real world. Alternatively, a computer onboard the tool controls the VLS during the additional optimization rounds while the user looks at the real world. In case of bad performance, the user has the option to restart the procedure, using the output of the just finished test as the initial estimate for the next test.

In some implementations, an optimization algorithm performed by the computer (when controlling the testing, including controlling the variable lens system and other user interfaces) searches for the maximum value of refraction (the more positive the better) in which the user reaches a 20/20 vision or better. The testing procedure stops regarding a specific refractive attribute (e.g. spherical power) when it reaches one of the following conditions: (a) the number of iterations passes a specified threshold; (b) there are no more alternatives of the refractive attribute to be tested; or (c) the entire set of available incremental changes from a given value of the refractive attribute have been tested, and none of these incremental changes produce an improvement in vision (as reported by the user). In some cases, if the testing procedure would otherwise stop because condition (c) is reached, then the last and penultimate settings are repeated in order to confirm the result. If this confirmation round confirms the result, the testing procedure stops; if it does not confirm the result, then the testing procedure continues.

In some implementations of this invention, methods to estimate a prescription include: (a) reducing spherical power if the refraction is overcorrecting, (b) increasing spherical power if the refraction does not reach the necessary power, (c) reducing or increasing cylindrical power if the astigmatism power changed or if a previous optometrist opted for not correcting astigmatism, or (d) changing an axis of astigmatism to improve the alignment of the glasses to the eye's astigmatic axis.

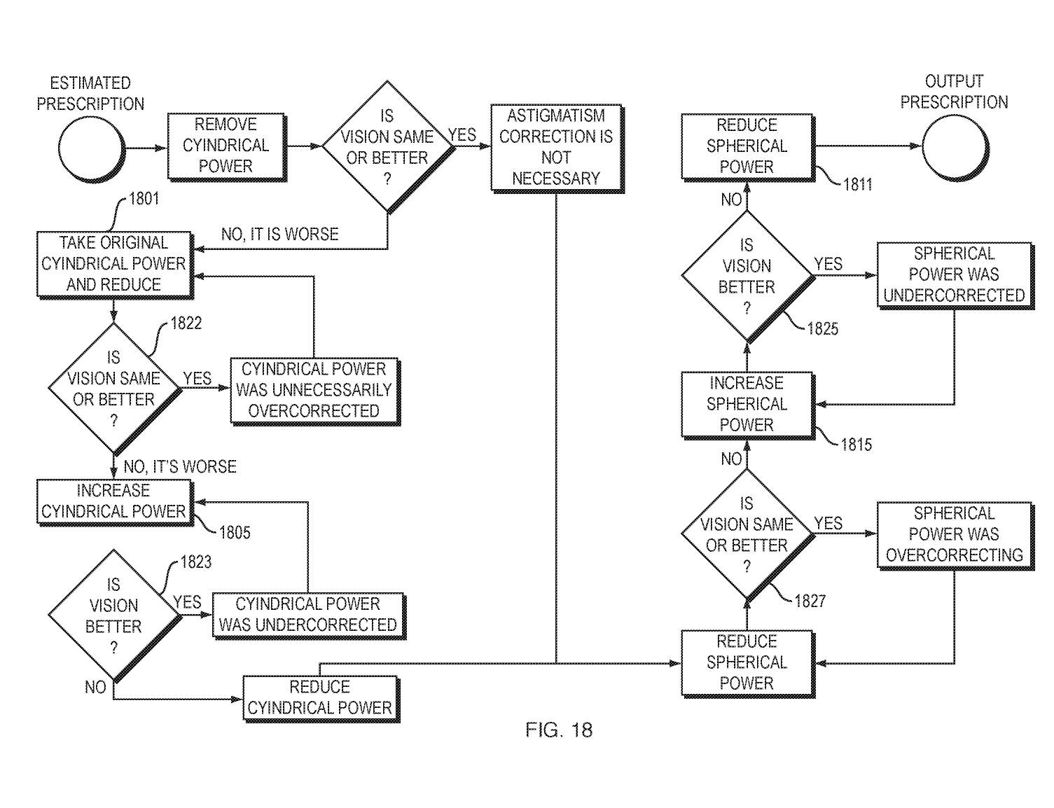

FIG. 18 is a flowchart that shows an example of a method for determining the best spherocylindrical refractive correction, in an illustrative implementation of this invention. The method includes steps in which adjustments are made to refractive attributes of a variable lens, such as reducing cylindrical power 1801, increasing cylindrical power 1805, reducing spherical power 1811, and increasing spherical power 1815. After each adjustment is made, feedback is received from the user regarding the visual effect of the adjustment (e.g., feedback received at steps 1822, 1823, 1825, 1827). For example, the feedback may indicate whether an adjustment (a) made an image perceived by the user clearer, (b) made an image perceived by the user less clear, or (c) made no difference.

In some implementations, after the optimization procedure would otherwise halt, the system tests for over-corrections by placing the focal power slightly outside what is estimated to be the focusing range of the user. If a new vision assessment is performed and the acuity is the same, the previous correction was not ideal and a new best refraction has been found. If the new acuity is worse, the previous refraction was better, confirming the correct focusing range of the user.

In the example shown in FIG. 18, a test is performed to determine if cylinder correction is needed, and if so, at which cylindrical power. For example, in some implementations, the user turns a knob on the tool to adjust between the measured refraction and an adjusted refraction without the cylindrical power. The user turns the knob to alternate between different refractions. Once the best option is chosen, the user presses a button on the bi-ocular tool. A computer (e.g., onboard the MCD or the tool) captures the button press, stores the preference and moves to the next state of the optimization flow.

FIG. 19 shows a method that involves iterative tests with user input, in an illustrative implementation of this invention. In the example shown in FIG. 19, a method includes the following steps: A user holds a refractive measurement tool up to his or her eyes, and looks through the tool at a near or far scene. The tool measures refractive aberrations of the human eye, and includes a variable lens system (VLS). One or more refractive attributes (e.g., spherical power, cylindrical power, cylindrical axis, prism or base) of the VLS are adjustable (Step 1901). Iterative vision tests are performed, in which refractive properties of the VLS are changed from iteration to iteration (Step 1902). I/O devices receive input from the user regarding which VLS setting results in clearer vision. For example, in some use scenarios, if spherical power is being optimized during a particular step of the testing procedure, the user inputs feedback regarding whether a test image appears clearer with the current VLS setting (a changed spherical power) than with the last VLS setting (a prior spherical power) (Step 1903). A computer analyzes data gathered in these tests, and calculates a refractive assessment. The refractive assessment specifies one or more refractive attributes (e.g., spherical power, cylindrical power, cylindrical axis, prism or base) for eyeglasses or contact lenses that would correct refractive aberrations of the user's right and left eyes, respectively (Step 1904). The refractive assessment is outputted, in human perceptible form, via one or more I/O devices (Step 1905).

FIG. 20 shows a method in which testing with the MCD attached is followed by testing with the MCD removed, in an illustrative implementation of this invention. In the example shown in FIG. 20, a method includes the following steps: The user removes the MCD from the refractive measurement tool, holds the tool up to the eye-level again, and looks out at a distant scene (preferably, at least three meters from the user). With the MCD removed, light from the distant scene travels in an optical path that passes through the tool: light travels from the scene, then through the tool's variable lens system, then through the tool's viewport, and then to the eyes (Step 2001). With the MCD removed, further trials are conducted, in which settings (e.g., spherical power, cylindrical power, cylindrical axis, prism or base) of the VLS are changed from trial to trial, and the user subjectively reports, after each new trial, whether the image looks clearer in the current trial than in the previous trial (Step 2002).

In some implementations, an eye's refractive aberrations for both near vision and distance vision are assessed when the MCD is attached to the front of the tool (e.g., such that the actual distance from the eye to the MCD is short).

FIG. 21 shows a method in which cues are displayed to control accommodation, in an illustrative implementation of this invention. In the example shown in FIG. 21, a method includes the following steps: The MCD screen displays visual cues to control accommodation and convergence of the test eye, such that the eye being tested focuses and converges as if looking at different distances (e.g., as if focusing at a short distance, or “at infinity”, or even optically “beyond infinity”). During these vision tests at different apparent distances, the refractive measurement tool gathers test data, which test data characterizes the full accommodation range of the eyes (Step 2101). A user inputs, via one or more I/O devices onboard the tool or MCD, the user's preferred viewing distances (e.g., close-up reading distance or mid-range distance for reading a computer screen) (Step 2102). A computer uses the test data in order to compute a refractive correction for the preferred viewing distances (Step 2103).

FIG. 22 shows a method in which initial settings of a VLS are based on prior knowledge, in an illustrative implementation of this invention. In the example shown in FIG. 22, a method includes the following steps: A computer onboard the tool or MCD calculates initial settings of the variable lens system (VLS) by using prior knowledge, such as data inputted by the user or obtained from a database. For example, in some cases, the user's last eye prescription is used as the starting point, so that the initial spherical power, cylindrical power, cylindrical axis, prism and base of the VLS are set in accordance with the last prescription (Step 2201). Then further trials are conducted, varying the VLS settings, to determine how the user's eye has changed since the last prescription (Step 2202).

In some cases, the prior knowledge includes data regarding refractive aberrations of the eye (such as the user's last eye prescription).

In some cases, a user inputs the prior knowledge (e.g., the user's most recent eyeglasses prescription or contacts lenses prescription) via one or more I/O devices onboard the tool or the MCD. In other cases, a computer onboard the tool or MCD accesses (e.g., via a local or global network, such as the Internet) an external database to obtain the prior knowledge. In other cases, the prior knowledge comprises data outputted by an objective or subjective measurement device onboard the refractive measurement tool or MCD, and the computer uses this data in order to calculate the initial VLS settings.

In other use scenarios, the initial settings of the variable lens system (VLS) are determined without the benefit of prior knowledge regarding refractive aberrations of the eye (e.g., without the last eyeglass prescription). In some cases, in the absence of this prior knowledge, a single VLS setting is used as common starting point for all users. In some cases, even if prior knowledge regarding refractive aberrations is not known, other knowledge is available that provides some guidance for the initial setting of the VLS. For example, in some implementations, a lookup table stored in electronic memory (onboard the MCD or refractive measurement tool) contains common or average refractive assessments for different age groups. If no prior knowledge is available, then the user inputs her or her age, and a computer determines, based on the lookup table, an age-appropriate starting setting for the VLS.

In some cases, different initial VLS settings are used for near vision testing and far distance testing.

In some cases, a computer calculates an adjustment to the VLS settings based on user preference data or medical data, and outputs control signals to cause this adjustment to occur. For example, in some cases: (a) the user preference data includes preferred test images, a preferred way to use reading material (such as mobile phones and newspapers), driving needs, gaming activity and working conditions; and (b) the medical information comprises data regarding health conditions, past medical interventions and current medication. This user preference data and medical information is either (a) stored in an electronic memory device onboard the MCD or refractive measurement tool, (b) inputted by a human user via an I/O device onboard the MCD or tool, or (c) accessed (via a wired or wireless communication link with the Internet) from a database stored on a remote computer.

FIG. 23 shows a method that includes taking photographs of different regions of an eye at different VLS settings, in an illustrative implementation of this invention. In the example shown in FIG. 23, a method includes the following steps: A camera onboard the MCD takes photographs of a region of a human eye, while refractive properties of the VLS are varied, such that different photographs are taken at different VLS settings (Step 2301). A computer calculates, based at least in part on the photographs, a 2D or 3D estimate of a structure that is located in that eye region (Step 2302)

In some use scenarios, the structure that is photographed is a cataract, other visual occluder in the eye, or a retinal deformation. For example, in some cases, the region that is photographed comprises at least part of the anterior segment of an eye or of the posterior segment of an eye. In some cases: (a) multiple light sources (in different positions with different emitting light wavelengths) illuminate the eye while the photographs are taken; and (b) the light sources are controlled by a computer onboard the refractive measurement tool or onboard the MCD.

In some implementations, a test for the cylindrical axis (also known as the axis of astigmatism) is performed.

FIG. 24 shows a method that involves rotating an axis of astigmatism, in an illustrative implementation of this invention. In the example shown in FIG. 24, a method includes the following steps: The user rotates a dial or knob on the tool to rotate the axis of astigmatism of the VLS. The user rotates the knob or dial and stops at the sharpest image that he or she sees (Step 2401). While the user makes these adjustments, the MCD screen displays a visual test pattern or sequence of visual test patterns (e.g., eye charts, pictures or videos) (Step 2402).

It is desirable, in some cases, when testing for refractive aberration of an eye, to control the accommodation (or inversely, the relaxation) of the eye. For example, when testing for distance vision, it is desirable for the eye muscles to be fully relaxed, reaching the furthest point the testing eye can focus. Often, the best correction for distance vision is the one that corrects views that are “at infinity” optically. In this way, when wearing eyeglasses that have the optimized prescription, the user will focus from the closest possible range to optical infinity, and not beyond optical infinity.

It is also desirable, in some cases, when testing for refractive aberration of an eye, to control convergence. In some cases, in a bi-ocular design, the visual stimuli presented to an idle eye is duplicated to the testing eye to create a stereo view and help to relax the eye lens of each eye through the convergence or divergence of the eyes. When the eyes converge to a closer point, the eyes tend to accommodate to focus at that spatial depth. When the eyes converge to infinity (looking straight), the eyes tend to focus at infinity, relaxing their ciliary muscles, and creating a favorable condition for measuring myopia. Convergence is correlated with accommodation/relaxation, but if the image is optically beyond infinity, the eye relaxes to focus beyond infinity even if convergence is to infinity only.

FIG. 25 shows a method that involves adjusting both spherical and cylindrical refractive attributes and controlling accommodation, in an illustrative implementation of this invention. In the example shown in FIG. 25, a method includes the following steps: The VLS settings are varied, such that both spherical and cylindrical refractive attributes of the VLS change simultaneously, for both eyes (Step 2501). After the estimated refraction is applied to the VLS (including sphere, cylinder axis, prism and base if needed), the spherical power of the portion of the VLS is increased so as to move a virtual object to a virtual position just beyond the farthest focal point that each eye, respectively, is able to see (Step 2502). Even if the image is never in focus, the visual cortex adjusts accommodation to the sharpest image, relaxing the eyes even more. Because the cylindrical component is applied to correct the vision of the patient, the eyes' focusing abilities are enhanced, leading to a better relaxation control than just setting the spherical power (Step 2503).

In some implementations, convergence of the eyes is controlled by (a) adjusting the distance between the right and left lenses sets of the VLS to match the user's pupillary distance, and (b) by changing the distance between the patterns on screen (Step 2601).

For example, refractive corrections for near vision may be optimized by controlling accommodation.

FIG. 26 shows a method that involves controlling convergence, in an illustrative implementation of this invention. In the example shown in FIG. 26, a method includes the following steps: By moving the right and left lenses of the VLS closer to each other, the refractive measurement tool forces the eyes to converge and accommodate to a closer object. The VLS is set to a stronger spherical power, making the user focus closer (Step 2601). At a desired reading distance, test iterations are performed in which spherical power is added, and visual acuity is retested (Step 2602). A computer onboard the MCD or refractive measurement tool analyzes the results, and estimates an optimized spherocylindrical correction for reading glasses (Step 2603).

### Virtual Reality

In illustrative implementations, the iterative testing procedure sometimes lasts for an extended period of time. In some cases, to keep the user engaged and happy, the vision testing is implemented in a game-like procedure. As the user advances stages in the game-like procedure, the refraction is being optimized. At the end of the testing procedure, the result is outputted on the screen.

In some implementations, after game-like testing ends, the refractive measurement tool (including the attached MCD) functions as virtual reality (VR) display. The VR display compensates for refractive errors not only for distance, but also for near view.

In some implementations, the VR display produces 3D imagery already corrected for the user's refractive error. The illusion of depth is achieved by changing convergence and accommodation as needed for the media being watched, but also correcting for the distance and near refractive error of the user. The placement of a virtual object in the 3D virtual world is translated into the spherical diopter power for the VLS by using the following equation: E=X+1/L−1/O, where O is the virtual distance in meters between the user's eye and the virtual object, and E, X and L are as described above. The new convergence of images after applying the refractive error is set to the distance 1/E from the user. If the range of 3D effects in diopters is bigger than user's focal range, the 3D effects are linearly scaled to match the user's focal range.

### Actuators

In illustrative implementations, each actuator (including each actuator for actuating any movement) is any kind of actuator, including a linear, rotary, electrical, piezoelectric, electro-active polymer, mechanical or electro-mechanical actuator. In some cases, the actuator includes and is powered by an electrical motor, including any stepper motor or servomotor. In some cases, the actuator includes a gear assembly, drive train, pivot, joint, rod, arm, or other component for transmitting motion. In some cases, one or more sensors are used to detect position, displacement or other data for feedback to one of more of the actuators. In some cases, an actuator comprises a mechanical actuator powered by motion imparted by a human (e.g., a dial, knob, button or slider that imparts motion, via a gear assembly, to one or more hardware components).

### Computers

In exemplary implementations of this invention, one or more electronic computers (e.g. 214, 219) are programmed and specially adapted: (1) to control the operation of, or interface with, hardware components of a refractive measurement tool (including any variable lens system, actuator, sensor, or I/O device) or of an MCD (including any display screen, microphone, speaker, camera, or I/O device); (2) to control one or more user interfaces during iterative vision tests, including any visual, auditory or haptic user interface onboard the refractive measurement tool or MCD; (3) analyzing data representing human input, including input regarding a user's visual perceptions during a vision test; (4) analyzing other data regarding the vision test gathered or outputted by the refractive measurement tool or MCD; (5) computing a refractive assessment for an eye, including calculating one or more refractive attributes (e.g., spherical power, cylindrical power, cylindrical axis, prism, base or BVD) of a human eye, based on user input and other data gathered during iterative eye tests, (6) to perform any other calculation, computation, program, algorithm, or computer function described or implied above; (7) to receive signals indicative of human input; (8) to output signals for controlling transducers for outputting information in human perceivable format; and (9) to process data, to perform computations, to execute any algorithm or software, and to control the read or write of data to and from memory devices (items 1-9 of this sentence referred to herein as the “Computer Tasks”).

Depending on the particular implementation of this invention, the one or more computers that perform these computational tasks are either located entirely onboard the MCD, or entirely onboard the refractive measurement tool, or distributed such that some are onboard the MCD and some are onboard the tool.

In some implementations, an electronics module (EM) of the refractive measurement tool includes one or more electronic computers (e.g., an integrated circuit, controller, microcontroller, field programmable gate array or other electronic processor). In some cases, these computers in the tool control the variable lens system (VLS), including while the MCD is attached to, or detached from, the tool.

In other cases, all or some of the computers that perform these computational tasks (including controlling the variable lens system) are located onboard the MCD. Control signals from these MCD processors are transmitted via a wired connection when the MCD is attached to the tool, and via a wireless connection when the MCD is not attached to the refractive measurement tool. If wireless control signals are used, then the tool includes a wireless communication module (including an antenna and wireless receiver, transceiver or transmitter) for receiving wireless control signals from the MCD. More generally, the one or more computers may be in any position or positions within or outside of the refractive measurement tool or MCD. For example, in some cases (a) at least one computer is housed in (or together with other components of) the refractive measurement tool or MCD, and (b) at least one computer is remote from other components of the refractive measurement tool or MCD.

Regardless of where the computers are located (whether onboard the MCD or the tool), they exercise indirect control over the variable lens system. The one or more computers output control signals to control components of an EM that in turn control a variable lens system (VLS). For example, in some cases, an EM controls a VLS by controlling current or voltage applied to an actuator or by controlling voltage applied to a liquid lens.

The one or more computers may be connected to each other or to other components in the refractive measurement tool or MCD either: (a) wirelessly, (b) by wired connection, (c) by fiber-optic link, or (d) by a combination of wired, wireless or fiber optic links.

In exemplary implementations, one or more computers are programmed to perform any and all calculations, computations, programs, algorithms, computer functions and computer tasks described or implied above. For example, in some cases: (a) a machine-accessible medium has instructions encoded thereon that specify steps in a software program; and (b) the computer accesses the instructions encoded on the machine-accessible medium, in order to determine steps to execute in the program. In exemplary implementations, the machine-accessible medium comprises a tangible non-transitory medium. In some cases, the machine-accessible medium comprises (a) a memory unit or (b) an auxiliary memory storage device. For example, in some cases, a control unit in a computer fetches the instructions from memory.

In illustrative implementations, one or more computers execute programs according to instructions encoded in one or more tangible, non-transitory, computer-readable media. For example, in some cases, these instructions comprise instructions for a computer to perform any calculation, computation, program, algorithm, or computer function described or implied above. For example, in some cases, instructions encoded in a tangible, non-transitory, computer-accessible medium comprise instructions for a computer to perform the Computer Tasks.

### Definitions

The terms “a” and “an”, when modifying a noun, do not imply that only one of the noun exists.

“Accommodation limit” of an eye means the furthest optical distance at which the eye is able to focus.

Here are some non-limiting examples of a “camera”: (a) a digital camera; (b) a digital grayscale camera; (c) a digital color camera; (d) a video camera; (e) a light sensor or image sensor, (f) a set or array of light sensors or image sensors; (g) an imaging system; (h) a light field camera or plenoptic camera; (i) a time-of-flight camera; and (j) a depth camera. A camera includes any computers or circuits that process data captured by the camera.

The term “comprise” (and grammatical variations thereof) shall be construed as if followed by “without limitation”. If A comprises B, then A includes B and may include other things.

The term “computer” includes any computational device that performs logical and arithmetic operations. For example, in some cases, a “computer” comprises an electronic computational device, such as an integrated circuit, a microprocessor, a mobile computing device, a laptop computer, a tablet computer, a personal computer, or a mainframe computer. In some cases, a “computer” comprises: (a) a central processing unit, (b) an ALU (arithmetic logic unit), (c) a memory unit, and (d) a control unit that controls actions of other components of the computer so that encoded steps of a program are executed in a sequence. In some cases, a “computer” also includes peripheral units including an auxiliary memory storage device (e.g., a disk drive or flash memory), or includes signal processing circuitry. However, a human is not a “computer”, as that term is used herein.

“Defined Term” means a term or phrase that is set forth in quotation marks in this Definitions section.

For an event to occur “during” a time period, it is not necessary that the event occur throughout the entire time period. For example, an event that occurs during only a portion of a given time period occurs “during” the given time period.

The term “e.g.” means for example.

The fact that an “example” or multiple examples of something are given does not imply that they are the only instances of that thing. An example (or a group of examples) is merely a non-exhaustive and non-limiting illustration.

Unless the context clearly indicates otherwise: (1) a phrase that includes “a first” thing and “a second” thing does not imply an order of the two things (or that there are only two of the things); and (2) such a phrase is simply a way of identifying the two things, respectively, so that they each may be referred to later with specificity (e.g., by referring to “the first” thing and “the second” thing later). For example, unless the context clearly indicates otherwise, if an equation has a first term and a second term, then the equation may (or may not) have more than two terms, and the first term may occur before or after the second term in the equation. A phrase that includes a “third” thing, a “fourth” thing and so on shall be construed in like manner.

“For instance” means for example.

“EPM Patent Application” is defined in the Summary section, above.

“EyeNetra Photorefraction Measurement” and “EPM” are defined in the Summary section, above.

“EPM aberrometer” and “EyeNetra Photorefraction Aberrometer” are defined in the Summary section, above.

“Herein” means in this document, including text, specification, claims, abstract, and drawings.

As used herein: (1) “implementation” means an implementation of this invention; (2) “embodiment” means an embodiment of this invention; (3) “case” means an implementation of this invention; and (4) “use scenario” means a use scenario of this invention.

The term “include” (and grammatical variations thereof) shall be construed as if followed by “without limitation”.

To “integrate” means either (a) to perform integration in the calculus sense, or (b) to compute a sum of discrete samples.

“Intensity” means any measure of or related to intensity, energy or power. For example, the “intensity” of light includes any of the following measures: irradiance, spectral irradiance, radiant energy, radiant flux, spectral power, radiant intensity, spectral intensity, radiance, spectral radiance, radiant exitance, radiant emittance, spectral radiant exitance, spectral radiant emittance, radiosity, radiant exposure or radiant energy density.

“I/O device” means an input/output device. Non-limiting examples of an I/O device include any device for (a) receiving input from a human user, (b) providing output to a human user, or (c) both. Non-limiting examples of an I/O device also include a touch screen, other electronic display screen, keyboard, mouse, microphone, handheld electronic game controller, digital stylus, display screen, speaker, or projector for projecting a visual display.

“Lens” means a single lens, compound lens or set of lenses.

“Light” means electromagnetic radiation of any frequency. For example, “light” includes, among other things, visible light and infrared light. Likewise, any term that directly or indirectly relates to light (e.g., “imaging”) shall be construed broadly as applying to electromagnetic radiation of any frequency.

The term “mobile computing device” or “MCD” means a device that includes a computer, a camera, a display screen and a wireless transceiver. Non-limiting examples of an MCD include a smartphone, cell phone, mobile phone, tablet computer, laptop computer and notebook computer.

“NETRA aberrometer” and “Near Eye Aberrometer” are defined in the Summary section, above.

“NETRA Measurement” and “Near Eye Measurment” are defined in the Summary section, above.

“NETRA Patent” is defined in the Summary section, above.

An “objective” measurement means a measurement that does not require feedback from a human user regarding the user's subjective visual perceptions.

Non-limiting examples of an “optical component” include an object that refracts light (such as a lens or prism), an object that reflects light (such as a mirror or beamsplitter), and an object that transmits or modulates light (such as a spatial light modulator).

The term “or” is inclusive, not exclusive. For example, A or B is true if A is true, or B is true, or both A or B are true. Also, for example, a calculation of A or B means a calculation of A, or a calculation of B, or a calculation of A and B.

A parenthesis is simply to make text easier to read, by indicating a grouping of words. A parenthesis does not mean that the parenthetical material is optional or may be ignored.

A sentence that says that an optical path “passes through” a set of multiple objects and that lists the objects in a particular order, does not imply that light passes through the objects in the particular order and does not imply any order in which the objects are positioned along the optical path. For example, to say that an optical path “passes through” A, B and C does not imply that light which travels along the optical path passes through A before B, and does not imply that B is positioned between A and C along the optical path.

Non-limiting examples of “refractive attributes” of a lens include spherical power, cylindrical power, and cylindrical axis. Non-limiting examples of “refractive attributes” of a refractive correction include spherical power, cylindrical power, cylindrical axis, prism, base and back vertex distance.

“Relaxation” of an eye means accommodation of an eye. The more relaxed an eye, the greater the optical distance at which the eye is focused.

As used herein, the term “set” does not include a group with no elements. Mentioning a first set and a second set does not, in and of itself, create any implication regarding whether or not the first and second sets overlap (that is, intersect).

“Some” means one or more.

“Spatial light modulator” and “SLM” each mean a device (i) that transmits light through the device or reflects light from the device, and (ii) that causes a modulation of the intensity, frequency, phase or polarization state of light transmitted through or reflected from the device, such that the modulation depends on the spatial position at which the light is incident on the device.

“Spherocylindrical attributes” means spherical power, cylindrical power and cylindrical axis. To say that a VLS is “spherocylindrical” means that the VLS has an adjustable spherical power, an adjustable cylindrical power and an adjustable cylindrical axis.

A “subjective” measurement means a measurement that requires feedback from a human user regarding the user's subjective visual perceptions.

As used herein, a “subset” of a set consists of less than all of the elements of the set.

“Substantially” means by at least ten percent. For example: (a) 112 is substantially larger than 100; and (b) 108 is not substantially larger than 100.

The term “such as” means for example.

To say that a machine-readable medium is “transitory” means that the medium is a transitory signal, such as an electromagnetic wave.

“Variable lens system” and “VLS” each mean a lens system that is configured to undergo variation in one or more refractive attributes of the lens system, such that: (a) the variation is due, at least in part, to either (i) a change in curvature of at least one lens in the lens system or (ii) motion of two or more lenses in the lens system relative to each other; and (b) the variation is not due, in whole or part, to removing a given lens from the lens system and replacing the given lens with a different lens.

“Variable lens system measurement” and “VLS subjective measurement” each mean a subjective measurement, in which a user provides input regarding the user's visual perceptions of light that passes through a variable lens system.

Except to the extent that the context clearly requires otherwise, if steps in a method are described herein, then the method includes variations in which: (1) steps in the method occur in any order or sequence, including any order or sequence different than that described; (2) any step or steps in the method occurs more than once; (3) different steps, out of the steps in the method, occur a different number of times during the method, (4) any combination of steps in the method is done in parallel or serially; (5) any step or steps in the method is performed iteratively; (6) a given step in the method is applied to the same thing each time that the given step occurs or is applied to different things each time that the given step occurs; or (7) the method includes other steps, in addition to the steps described.

Saying that A and B each mean X is the same as saying that A means X and B means X.

This Definitions section shall, in all cases, control over and override any other definition of the Defined Terms. For example, the definitions of Defined Terms set forth in this Definitions section override common usage or any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. To the extent that any term or phrase is defined or clarified herein, such definition or clarification applies to any grammatical variation of such term or phrase, taking into account the difference in grammatical form. For example, the grammatical variations include noun, verb, participle, adjective, and possessive forms, and different declensions, and different tenses. In each case described in this paragraph, the Applicant or Applicants are acting as his, her, its or their own lexicographer.

### Variations

This invention may be implemented in many different ways. Here are some non-limiting examples:

In some implementations, the refractive measurement tool, with the MCD attached, is used to perform other cognitive tests on the user, including duochrome analysis, Amsler grid, cataract screenings, and binocularity tests.

In some implementations: (a) the refractive measurement tool 201 measures refractive aberrations of an eye; and (b) additional apparatus 228 for measuring other conditions of the eye are also housed in the tool 201. For example, in some cases, the additional apparatus 228 comprises additional cameras and sensors for retinal imaging, Scheimpflug imaging, Purkinje imaging, tonometry or corneal topography. In some cases, the accuracy of readings by this additional apparatus 228 are improved by adjusting refractive attributes of the VLS (e.g., focal length) and by taking into account subjective measurements that involve the VLS.

In some implementations, this invention is a method comprising, in combination: (a) attaching a mobile computing device to a tool, which tool includes a variable lens system, a spatial light modulator, and an additional lens; (b) positioning the tool such that light from a display screen of the mobile computing device travels in an optical path from the display screen to an eye of a user, which optical path passes through the variable lens system, the spatial light modulator and the additional lens; (c) taking variable lens system measurements; and (d) taking NETRA Measurements. In some cases: (a) a computer calculates, based on the variable lens system measurements, a first setting of the variable lens system, which first setting comprises one or more spherocylindrical attributes of the variable lens system; and (b) one or more spherocylindrical attributes of the variable lens system are at the first setting when at least some of the NETRA Measurements are taken. In some cases: (a) the eye has an accommodation limit; and (b) the first setting comprises spherocylindrical attributes of the variable lens system that tend to relax the eye to the accommodation limit. In some cases: (a) a set of one or more of the NETRA Measurements are taken while the variable lens system is at a first spherical power; (b) one or more computers calculate, based on the set of one or more NETRA Measurements, a refractive correction for the eye, which refractive correction is at second spherical power; (c) the one or more computers calculate an other refractive correction for the eye, which other refractive correction is at a third spherical power; and (d) the third spherical power is equal to the first spherical power minus the second spherical power. In some cases: (a) the spatial light modulator includes a first region, second region and third region; and (b) the light is filtered as it passes through the spatial light modulator, such that upon exiting the first, second and third regions, respectively, the light has a first color spectrum, second color spectrum and third color spectrum, the first, second and third color spectrums being different from each other. In some cases, the method further comprises: (a) taking NETRA Measurements at a first angular orientation of the spatial light modulator; and (b) taking NETRA Measurements at a second angular orientation of the spatial light modulator, the first and second angular orientations being different from each other. In some cases: (a) one or more computers calculate, based on the NETRA Measurements, a specific setting of refractive attributes of the variable lens system; and (b) the display screen displays virtual reality content while refractive attributes of the variable lens system are at the specific setting. Each of the cases described above in this paragraph is an example of the method described in the first sentence of this paragraph, and is also an example of an embodiment of this invention that may be combined with other embodiments of this invention.

In some implementations, this invention is a tool that comprises a variable lens system, a spatial light modulator, and an additional lens, wherein the tool is configured to be attached to a mobile computing device, such that (a) light from a display screen of the mobile computing device travels in an optical path from the display screen to an eye of a user, which optical path passes through the variable lens system, the spatial light modulator and the additional lens; and (b) the tool and mobile computing device together take variable lens system measurements and NETRA Measurements. In some cases, the spatial light modulator comprises a static mask. In some cases: (a) the spatial light modulator includes a first region, second region and third region; and (b) the first, second and third regions comprise filters for filtering light such that light exiting the first, second and third regions has a first color spectrum, second color spectrum and third color spectrum, respectively, the first, second and third color spectrums being different from each other. In some cases, the tool includes one or more actuators for rotating the spatial light modulator, such that: (a) a first set of the NETRA Measurements are taken at a first angular orientation of the spatial light modulator; and (b) a second set of the NETRA Measurements are taken at a second angular orientation of the spatial light modulator, the first and second angular orientations being different from each other. Each of the cases described above in this paragraph is an example of the tool described in the first sentence of this paragraph, and is also an example of an embodiment of this invention that may be combined with other embodiments of this invention.

In some implementations, this invention is a method comprising, in combination: (a) attaching a mobile computing device to a tool, which tool includes a variable lens system, a spatial light modulator, an additional lens, and a light source; (b) positioning the tool such that light travels in an optical path from a light source to an eye of a user and then from the eye to a camera, which optical path passes at least once through the variable lens system, the spatial light modulator and the additional lens; (c) taking variable lens system measurements; and (d) taking EyeNetra Photorefraction Measurements. In some cases: (a) one or more computers calculate, based on the EyeNetra Photorefraction Measurements, a specific setting of refractive attributes of the variable lens system; and (b) the display screen displays virtual reality content while refractive attributes of the variable lens system are at the specific setting. In some cases: (a) a computer calculates, based on the variable lens system measurements, a specific setting of the variable lens system, which specific setting comprises one or more spherocylindrical attributes of the variable lens system; and (b) one or more spherocylindrical attributes of the variable lens system are at the specific setting when at least some of the EyeNetra Photorefraction Measurements are taken. In some cases: (a) the eye has an accommodation limit; and (b) the specific setting comprises spherocylindrical attributes of the variable lens system that tend to relax the eye to the accommodation limit. In some cases: (a) a set of one or more of the EyeNetra Photorefraction Measurements are taken while the variable lens system is at a first spherical power; (b) one or more computers calculate, based on the set of one or more EyeNetra Photorefraction Measurements, a refractive correction for the eye, which refractive correction is at second spherical power; (c) the one or more computers calculate another refractive correction of the eye, which other refractive correction is at a third spherical power; and (d) the third spherical power is equal to the first spherical power minus the second spherical power. In some cases:(a) spherical power of the variable lens system is changed from a first setting to a second setting; and (b) while the eye is changing focal length to accommodate to the second setting, multiple EyeNetra Photorefraction Measurements are taken to determine speed of accommodation of the eye. Each of the cases described above in this paragraph is an example of the method described in the first sentence of this paragraph, and is also an example of an embodiment of this invention that may be combined with other embodiments of this invention.

In some implementations, this invention is a tool that comprises a variable lens system, a spatial light modulator, and an additional lens, wherein the tool is configured to be attached to a mobile computing device, such that (a) light from a display screen of the mobile computing device travels in an optical path from a light source to an eye of a user and then from the eye to a camera, which optical path passes at least once through the variable lens system, the spatial light modulator and the additional lens; and (b) the tool and mobile computing device together take variable lens system measurements and EyeNetra Photorefraction Measurements. In some cases, the light source is housed in, and is part of, the tool. In some cases, the camera is housed in, and is part of, the mobile computing device. Each of the cases described above in this paragraph is an example of the tool described in the first sentence of this paragraph, and is also an example of an embodiment of this invention that may be combined with other embodiments of this invention.

The above description (including without limitation any attached drawings and figures) describes illustrative implementations of the invention. However, the invention may be implemented in other ways. The methods and apparatus which are described above are merely illustrative applications of the principles of the invention. Other arrangements, methods, modifications, and substitutions by one of ordinary skill in the art are therefore also within the scope of the present invention. Numerous modifications may be made by those skilled in the art without departing from the scope of the invention. Also, this invention includes without limitation each combination and permutation of one or more of the abovementioned implementations, embodiments and features.

What is claimed is:

1. A method comprising, in combination:
(a) attaching a mobile computing device to a tool, which tool includes a variable lens system, a spatial light modulator, and an additional lens;
(b) positioning the tool such that light from a display screen of the mobile computing device travels in an optical path from the display screen to an eye of a user, which optical path passes through the variable lens system, the spatial light modulator and the additional lens;
(c) taking variable lens system measurements; and
(d) taking NETRA Measurements.

2. The method of claim 1, wherein:
(a) a computer calculates, based on the variable lens system measurements, a first setting of the variable lens system, which first setting comprises one or more spherocylindrical attributes of the variable lens system; and
(b) one or more spherocylindrical attributes of the variable lens system are at the first setting when at least some of the NETRA Measurements are taken.

3. The method of claim 2, wherein:
(a) the eye has an accommodation limit; and
(b) the first setting comprises spherocylindrical attributes of the variable lens system that tend to relax the eye to the accommodation limit.

4. The method of claim 1, wherein:
(a) a set of one or more of the NETRA Measurements are taken while the variable lens system is at a first spherical power;
(b) one or more computers calculate, based on the set of one or more NETRA Measurements, a refractive correction for the eye, which refractive correction is at second spherical power;
(c) the one or more computers calculate an other refractive correction for the eye, which other refractive correction is at a third spherical power; and
(d) the third spherical power is equal to the first spherical power minus the second spherical power.

5. The method of claim 1, wherein:
(a) the spatial light modulator includes a first region, second region and third region; and
(b) the light is filtered as it passes through the spatial light modulator, such that upon exiting the first, second and third regions, respectively, the light has a first color spectrum, second color spectrum and third color spectrum, the first, second and third color spectrums being different from each other.

6. The method of claim 5, further comprising:
(a) taking NETRA Measurements at a first angular orientation of the spatial light modulator; and
(b) taking NETRA Measurements at a second angular orientation of the spatial light modulator, the first and second angular orientations being different from each other.

7. The method of claim 1, wherein:
(a) one or more computers calculate, based on the NETRA Measurements, a specific setting of refractive attributes of the variable lens system; and
(b) the display screen displays virtual reality content while refractive attributes of the variable lens system are at the specific setting.

8. A tool that comprises a variable lens system, a spatial light modulator, and an additional lens, wherein the tool is configured to be attached to a mobile computing device, such that
(a) light from a display screen of the mobile computing device travels in an optical path from the display screen to an eye of a user, which optical path passes through the variable lens system, the spatial light modulator and the additional lens; and
(b) the tool and mobile computing device together take variable lens system measurements and NETRA Measurements.

9. The tool of claim 8, wherein the spatial light modulator comprises a static mask.

10. The tool of claim 8, wherein:
(a) the spatial light modulator includes a first region, second region and third region; and
(b) the first, second and third regions comprise filters for filtering light such that light exiting the first, second and third regions has a first color spectrum, second color spectrum and third color spectrum, respectively, the first, second and third color spectrums being different from each other.

11. The tool of claim 10, wherein the tool includes one or more actuators for rotating the spatial light modulator, such that:
(a) a first set of the NETRA Measurements are taken at a first angular orientation of the spatial light modulator; and
(b) a second set of the NETRA Measurements are taken at a second angular orientation of the spatial light modulator, the first and second angular orientations being different from each other.

12. A method comprising, in combination:
(a) attaching a mobile computing device to a tool, which tool includes a variable lens system, a spatial light modulator, an additional lens, and a light source;
(b) positioning the tool such that light travels in an optical path from a light source to an eye of a user and then from the eye to a camera, which optical path passes at least once through the variable lens system, the spatial light modulator and the additional lens;
(c) taking variable lens system measurements; and
(d) taking EyeNetra Photorefraction Measurements.

13. The method of claim 12, wherein:
(a) one or more computers calculate, based on the EyeNetra Photorefraction Measurements, a specific setting of refractive attributes of the variable lens system; and
(b) the display screen displays virtual reality content while refractive attributes of the variable lens system are at the specific setting.

14. The method of claim 12, wherein:
(a) a computer calculates, based on the variable lens system measurements, a specific setting of the variable lens system, which specific setting comprises one or more spherocylindrical attributes of the variable lens system; and
(b) one or more spherocylindrical attributes of the variable lens system are at the specific setting when at least some of the EyeNetra Photorefraction Measurements are taken.

15. The method of claim 14, wherein:
(a) the eye has an accommodation limit; and
(b) the specific setting comprises spherocylindrical attributes of the variable lens system that tend to relax the eye to the accommodation limit.

16. The method of claim 12, wherein:
(a) a set of one or more of the EyeNetra Photorefraction Measurements are taken while the variable lens system is at a first spherical power;
(b) one or more computers calculate, based on the set of one or more EyeNetra Photorefraction Measurements, a refractive correction for the eye, which refractive correction is at second spherical power;
(c) the one or more computers calculate another refractive correction of the eye, which other refractive correction is at a third spherical power; and
(d) the third spherical power is equal to the first spherical power minus the second spherical power.

17. The method of claim 12, wherein:
(a) spherical power of the variable lens system is changed from a first setting to a second setting; and
(b) while the eye is changing focal length to accommodate to the second setting, multiple EyeNetra Photorefraction Measurements are taken to determine speed of accommodation of the eye.

18. A tool that comprises a variable lens system, a spatial light modulator, and an additional lens, wherein the tool is configured to be attached to a mobile computing device, such that
(a) light from a display screen of the mobile computing device travels in an optical path from a light source to an eye of a user and then from the eye to a camera, which optical path passes at least once through the variable lens system, the spatial light modulator and the additional lens; and
(b) the tool and mobile computing device together take variable lens system measurements and EyeNetra Photorefraction Measurements.

19. The tool of claim 18, wherein the light source is housed in, and is part of, the tool.

20. The tool of claim 18, wherein the camera is housed in, and is part of, the mobile computing device.
